(19) United States
(12) Patent Application Publication
(10) Pub. No.: US 2006/0064768 A1
(43) Pub. Date: Mar. 23, 2006

(54) Murine Pten null prostate cancer model

(75) Inventors: Hong Wu, Los Angeles, CA (US); Xin Lu, Los Angeles, CA (US); Shunyou Wang, Los Angeles, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/946,894

(22) Filed: Sep. 21, 2004

(51) Int. Cl. A01K 67/027

(52) U.S. Cl. 800/18

(57) ABSTRACT

The invention provides a transgenic mouse and cell lines with a homozygous disruption of a chromosomal PTEN gene in prostate cells. The mouse progresses from hyperplasia to metastatic cancer and can be used to identify prostate cancer therapeutics and genes that are differentially regulated during androgen dependent and androgen independent prostate cancer progression.

### FEDERALLY SPONSORED RESEARCH OR DEVELOPMENT

This invention was made with Government support of Grant No. DAMD17-00-1-0010, awarded by the Department of Defense and Grant Nos. CA84128 and CA92131 awarded by NIH. The Government has certain rights in this invention.

### CROSS-REFERENCES TO RELATED APPLICATIONS NOT APPLICABLE

### STATEMENT AS TO RIGHTS TO INVENTIONS MADE UNDER

### BACKGROUND OF THE INVENTION

Prostate cancer is the most common malignancy in men and the second leading cause of male cancer-related deaths in the Western world. Its development proceeds through a series of defined states, including prostatic intraepithelial neoplasia (PIN), prostate cancer in situ, invasive and metastatic cancer. The standard therapies include androgen ablation that initially causes tumor regression. However tumor cells will eventually relapse and develop into hormone refractory prostate cancer (HRPC)(Denis, L. et al., Cancer 72:3888-3895 (1993); Landis, S. H. et al., Cancer Statistics, Vol 49 (1999)).

The PTEN (phosphatase and tensin homologue deleted on chromosome 10) tumor suppressor gene is one of the most frequently mutated/deleted gene in various human cancers (Bose, S. et al., Hum Pathol 33:405-409 (2002); Deocampo, N. D. et al., Minerva Endocrinol 28:145-153 (2003); Sun, H. et al., Diagn. Mol. Pathol. 11:204-211 (2002); Wang, J. Y. et al., Virchows Arch. 442:437-443 (2003); Zhou, X. P. et al., Am. J. Pathol. 161:439-447 (2002)). Germ line mutations in the PTEN gene have been associated with Cowden syndrome and related diseases in which patients develop hyperplastic lesions (harmatomas) in multiple organs with increased risks of malignant transformation (Dahia, P. L. Cancer 7:115-129 (2000); Liaw, D. et al., Nat. Genet. 16:64-67 (1997); Marsh, D. J. et al.; Hum. Mol. Genel 8:1461-1472 (1999)). PTEN alteration is strongly implicated in prostate cancer development. PTEN deletions and/or mutations are found in 30% of primary prostate cancers (Dahia, P. L. Cancer 7:115-129 (2000); Sellers, W. A. et al., “Somatic Genetics of Prostate Cancer: Oncogenes and Tumore Suppressors” (Philadelphia: Lippincott Williams & Wilkins) (2002)) and 63% of metastatic prostate tissue samples (Suzuki, H. et al., Cancer Res. 58:204-209 (1998b)), placing PTEN mutation among the most common genetic alterations reported in human prostate cancers.

PTEN-controlled signaling pathways are frequently altered in human prostate cancers, making them promising targets for therapeutic strategies (DeMarzo, A. M. et al., Lancet 361:955-964 (2003); Sellers, W. A. et al., “Somatic Genetics of Prostate Cancer: Oncogenes and Tumore Suppressors” (Philadelphia: Lippincott Williams & Wilkins) (2002); Vivanco, I. et al., Nat. Rev. Cancer 2:489-501 (2002)). The major function of the tumor suppressor PTEN relies on its phosphatase activity and subsequent antagonism of the PI3K/AKT pathway (Cantley, L. C. et al., Proc Natl Acad Sci USA 96:4240-4245 (1999); Di Cristofano, A. et al., Cell 100:387-390 (2000); Meehama, T. et al., Annu. Rev. Biochem. 70:247-279 (2001)). Loss of PTENfunction, either in murine embryonic stem cells or in human cancer cell lines, results in accumulation of PIP3 and activation of its downstream effectors, such as AKT/PKB Stambolic, V., et al. Cell 95:29-39 (1998); Sun, H. et al., Proc. Natl. Acad. Sci. USA 96 96:6199-6204 (1999); Wu, X. et al., Proc. Natl. Acad. Sci. USA 95:15587-15591 (1998)). As a serine/threonine protein kinase, AKT functions by phosphorylating key intermediate signaling molecules, such as glycogen synthase kinase-3 (GSK3), BAD, Caspase 9, IκB, leading to increased cell metabolism, cell growth, and cell survival (Di Cristofano, A. et al., Cell 100:387-390 (2000); Hanahan, D. et al., Cell 100:57-70 (2000); Vivanco, I. et al., Nat. Rev. Cancer 2:489-501 (2002)). Recent studies also suggest that PTEN may function through AKT-independent mechanisms (Freeman, D. J. et al., Cancer Cell 3:117-130 (2003); Gao, X. et al., Dev Biol 221:404-418 (2000); Weng, L. et al., Hum. Mol. Genet. 10:237-242 (2001)).

Inactivation of Pten in mouse models has confirmed PTEN as a bona fide tumor suppressor. Pten^{+/-} mice showed a broad spectrum of spontaneous tumor development, with a bias towards organs such as large and small intestines, lymphoid, mammary, thyroid, endometrial, and adrenal glands (Di Cristofano, A. et al., Nat Genet 19:348-355 (1998); Podsypanina, K. et al., Proc. Natl. Acad. Sci. USA 96:1563-1568 (1999); Stambolic, V. et al., Cancer Res. 60:3605-3611 (2000); Suzuki, A. et al., Curr. Biol. 8:1169-1178 (1998a)). Since homozygous deletion of Pten causes early embryonic lethality, previous studies for prostate cancers caused by Pten deletion were invariably using Pten heterozygous mice. Different rates of prostatic hyperplasia and cancer have also been reported in the above studies. Our recent study demonstrated that Pten^{+/-} male mice on a Balb/c/129 genetic background develop lesions (PIN) with near 100% penetrance (Freeman, D. J. et al., submitted). However, the latency for PIN formation is rather long, approximately 10 months, and these PIN lesions never progress to metastatic disease.

Mice with combined deletion of Pten and other tumor suppressor genes, as possible “second hits”, have been generated. Pten^{+/-};p27^{-/-} mice develop prostate carcinoma within 3 months postnally with complete penetrance (Di Cristofano, A. et al., Nat Genet 27:222-224 (2001b)). Pten +/−;NKk×3.1^{-/-} compound mutant mice display an increased incidence of high grade PIN but not prostate cancer (Kim, M. J. et al., Proc Natl Acad Sci USA 99:2884-2889 (2002)). Similarly, Ink4a/Arf deficiency reduced tumor-free survival and shortened the latency of PIN associated with Pten heterozygosity (You, M. J. et al., Proc. Natl. Acad. Sci. USA 99:1455-1460 (2002)). However, no metastatic prostate cancers were reported in these models (Di Cristofano, A. et al., Nat Genet 27:222-224 (2001 a); Kim, M. J. et al., Proc Natl Acad Sci USA 99:2884-2889 (2002); You, M. J. et al., Proc. Natl. Acad. Sci. USA 99:1455-1460 (2002)). Pten heterozygous mice were also crossed with the well-characterized TRAMP model (Greenberg, N. M. et al., Proc Natl Acad Sci USA 92:3439-3443 (1995)). Pten LOH significantly shortened the average life span of TRAMP mice from 245 days to 159 days (Kwabi-Addo, B. et al., Proc Natl Acad Sci USA 98:11563-11568 (2001)). More recently, the MPAKT model was created which expressed constitutively activated AKT in mouse prostate epithelial cells (Majumder, P. K. et al., Proc. Natl. Acad. Sci. USA 100:7841-7846 (2003)). The MPAKT mice develop PIN lesions in the ventral prostate with prominent bladder obstruction. No progression to metastatic prostate cancer was reported (Majumder, P. K. et al., Proc. Natl. Acad. Sci. USA 100:7841-7846 (2003)).

Although genes that are involved in human prostate cancer have been identified, manipulation of those genes in model organisms has failed to generate a convenient model that closely mimics the progression of human prostate cancer. The present invention solves this and other needs.

### BRIEF SUMMARY OF THE INVENTION

In one aspect, the present invention provides a transgenic postnatal mouse that comprises a Pten-null prostate cell, wherein the Pten-null prostate cell comprises a genome comprising a homozygous disruption of the Pten gene, and wherein the Pten-null prostate cell has decreased levels of functional PTEN protein as compared to a prostate cell from a non-transgenic post-natal mouse.

In another aspect, the present invention provides a method of making a transgenic postnatal mouse of claim 1, the method comprising the steps of: crossing a first mouse comprising a Pten nucleic acid construct with a second mouse comprising a prostate-specific inducer of site-specific recombination. The Pten nucleic acid construct comprises a Pten nucleic acid comprising specific recombination sites, and in the absence of recombination, the Pten nucleic acid expresses a functional PTEN protein. After the cross is complete and progeny have been born, progeny that have a prostate-specific homozygous disruption of the Pten gene and decreased expression of functional PTEN protein in prostate cells are identified.

In one embodiment, the Pten nucleic acid construct comprises loxP sites that flank a region of the genomic Pten nucleic acid and the inducer of site-specific recombination comprises a Cre nucleic acid under the control of a prostate specific promoter. In a further embodiment, the loxP sites flank exon 5 of the genomic Pten nucleic acid. In yet another embodiment, the prostate specific promoter is a probasin promoter.

In another aspect, the present invention provides a method of stimulating the deregulated growth of prostate cells in a mouse, by providing the mouse described above, i.e., a transgenic postnatal mouse that comprises a Pten-null prostate cell, wherein the Pten-null prostate cell comprises a genome comprising a homozygous disruption of the Pten gene, and wherein the Pten-null prostate cell has decreased levels of functional PTEN protein as compared to a prostate cell from a non-transgenic post-natal mouse. The mouse is then allowed to grow until prostate cell hyperplasia is detected. The mouse can also be allowed to grow until other stages of deregulated growth of prostate cells are detected, e.g., prostatic intraepithelial neoplasia (PIN), invasive adenocarcinoma of the prostate, or metastatic prostate cancer. The mouse can also be allowed to grow until androgen independent cancer cells are detected.

In another aspect, the present invention provides method for assessing the effect of a composition or treatment on prostate cancer, by providing the mouse described above, i.e., a transgenic postnatal mouse that comprises a Pten-null prostate cell, wherein the Pten-null prostate cell comprises a genome comprising a homozygous disruption of the Pten gene, and wherein the Pten-null prostate cell has decreased levels of functional PTEN protein as compared to a prostate cell from a non-transgenic post-natal mouse. The mouse is then allowed to grow until prostate cancer is detected, and a test composition or treatment is applied to the mouse. After application, the effect of the composition or treatment on prostate cancer in the mouse is determined. The effect of a composition or treatment on a particular stage of prostate cancer can also be determined, e.g. an effect on prostatic intraepithelial neoplasia (PIN), an effect on invasive adenocarcinoma, or an effect on metastatic prostate cancer.

In another aspect the present invention provides a method for assessing the effect of a composition or treatment on androgen independent prostate cancer, by providing the mouse described above, i.e., a transgenic postnatal mouse that comprises a Pten-null prostate cell, wherein the Pten-null prostate cell comprises a genome comprising a homozygous disruption of the Pten gene, and wherein the Pten-null prostate cell has decreased levels of functional PTEN protein as compared to a.prostate cell from a non-transgenic post-natal mouse. The mouse is then allowed to grow until androgen independent prostate cancer is detected, and a test composition or treatment is applied to the mouse. After application, the effect of the composition or treatment on androgen independent prostate cancer in the mouse is determined. The mouse can be subjected to an androgen ablation therapy, e.g., a surgical treatment or chemical androgen ablation.

In another aspect, the present invention provides a method for identifying a prostate cancer biomarker, by providing the mouse described above, i.e., a transgenic postnatal mouse that comprises a Pten-null prostate cell, wherein the Pten-null prostate cell comprises a genome comprising a homozygous disruption of the Pten gene, and wherein the Pten-null prostate cell has decreased levels of functional PTEN protein as compared to a prostate cell from a non-transgenic post-natal mouse. The mouse is then allowed to grow until prostate cancer is detected, and an expression profile of a biological sample from the transgenic postnatal mouse is compared to an expression profile of a biological sample from a control postnatal mouse. Differences in expression profile that occur in the transgenic postnatal mouse relative to the control mouse, are then used to identify a prostate cancer biomarker. Similar methods can be used to identify biomarkers for a particular stage of prostate cancer, e.g. prostatic intraepithelial neoplasia (PIN), invasive adenocarcinoma, or metastatic prostate cancer.

In another aspect the present invention provides a method for identifying an androgen independent prostate cancer-biomarker, by providing the mouse described above, i.e., a transgenic postnatal mouse that comprises a Pten-null prostate cell, wherein the Pten-null prostate cell comprises a genome comprising a homozygous disruption of the Pten gene, and wherein the Pten-null prostate cell has decreased levels of functional PTEN protein as compared to a prostate cell from a non-transgenic post-natal mouse. The mouse is then allowed to grow until androgen independent prostate cancer is detected, and an expression profile of a biological sample from the transgenic postnatal mouse is compared to an expression profile of a biological sample from a control postnatal mouse. Differences in expression profile that occur in the transgenic postnatal mouse relative to the control mouse, are then used to identify an androgen independent prostate cancer biomarker. The mouse can be subjected to an androgen ablation therapy, e.g., a surgical treatment or chemical androgen ablation.

In a further aspect the present invention provides a Pten-null prostate cell, wherein a genome of the Pten-null prostate cell comprises a homozygous disruption of the Pten gene, and wherein the Pten-null prostate cell has decreased levels of functional PTEN protein as compared to a wild-type prostate cell. The Pten-null prostate cell can be isolated e.g., from the mouse described above. In one embodiment, the Pten-null prostate cell survives in the absence of androgens.

### BRIEF DESCRIPTION OF THE DRAWINGS

FIG. 1 provides evidence of a conditional deletion of the Pten tumor suppressor gene in prostate. Genomic DNA's were prepared from individual prostate lobe and indicted tissues of a 9 week old Pten^{loxp/+}; PB-Cre4⁺ male mouse PCR analysis shows Pten deletion (Δ5) is very prostate specific: except some leakage in the seminal vesicle, no Pten deletion can be detected in tissues other than the prostate.

FIG. 2 provides evidence of the response of Pten null prostate tumors to androgen ablation therapy. Top panel: 16 week old Pten conditional knockout mice (Mut) and their wild type litter mates were castrated for the indicated period, and prostate tissue were harvested for TUNEL analysis. Quantification is shown in the top panel, p<0.005. Bottom panel: Pten null prostate cancer cells remain proliferative in the absence of androgen. Tissue sections from the aforementioned animals were stained with anti-Ki67 antibody, an indicator of cell proliferation. Quantification is shown in the bottom panel, p<0.005.

FIG. 3 provides a list of the top 100 dysregulated genes that are expressed in Pten disrupted prostate cancer cells. TNAs extracted from four pairs of WT and Pten disrupted littermates were used for microarray analysis. Genes with significantly altered expression were identified by SAM analysis. For some genes, expression changes were further confirmed by Western blot or immunohistochemistry.

FIG. 4 provides a list of 1041 significantly altered genes/ESTs in that are expressed in Pten disrupted prostate cancer cells. Among them, 579 are up-regulated in Pten null cancer and 462 are down-regulated.

### DETAILED DESCRIPTION OF THE INVENTION

I. Introduction

Inactivation of the PTEN tumor suppressor gene is one of the most frequent genetic alterations found in human prostate cancers. Since loss of PTEN function causes embryonic lethality, Pten was specifically inactivated in the murine prostate gland. Surprisingly, the Pten prostate cancer model recapitulates the disease progression seen in humans: initiation of prostate cancer with prostatic intraepithelial neoplasia (PIN), followed by progression to invasive adenocarcinoma, and subsequent metastasis with defined kinetics. This is the first demonstration of a prostate cancer model that mimics the progression of human prostate cancer. Furthermore, while Pten null prostate cancers regress after androgen ablation, they are capable of proliferating in the absence of androgen. This provides the first example of androgen insensitive cancer in a model animal.

The transgenic mice that comprise a prostate specific Pten deletion can be used as a model of prostate cancer progression, e.g., either androgen dependent or androgen independent progression. Moreover, the transgenic mice that comprise a prostate specific Pten deletion can be used to assess therapeutic compositions and treatments for prostate cancer, including pharmaceutical compositions, chemotherapeutic agents, radiation therapy, and surgical treatments or combinations therein. Cells derived from prostate of transgenic mice that comprise a prostate specific Pten deletion can also be used to assess therapeutic agents for prostate cancer, e.g., using high throughput assays.

Inactivation of a Pten gene results in changes in expression of other nucleic acids and proteins. Such changes in expression indicate that a nucleic acid or protein is a biomarker of protate cancer. Transgenic mice that comprise a prostate specific Pten deletion or cells derived from such mice can also be used to identify biomarkers of prostate cancer, e.g., by determining the levels of an expression product of a nucleic acid, e.g., a messenger RNA, a protein, or a post-translationally modified protein.

II. Definitions

The term “transgenic animal” or “transgenic mouse” refers to an animal or mouse that contains within its genome a specific gene that has been disrupted by the method of gene targeting. The transgenic animal includes both the heterozygote animal (i.e., one defective allele and one wild-type allele) and the homozygous animal (i.e., two defective alleles).

The term, “transgenic cell”, refers to a cell containing within its genome a Pten gene that has been disrupted, modified, altered, or replaced completely or partially by the method of gene targeting.

A “Pten gene” refers to a nucleic acid that encodes a PTEN protein. In preferred embodiments, a Pten gene is a mouse Pten gene. The mouse PTEN protein sequence is found at Accession number O08586 and the encoding nucleic acid sequence is found at Accession number NM_{—}008960, each of which are herein incorporated by reference. The mouse Pten gene has been mapped to mouse chromosome 19 and the locus tag is MGI:109583, which is also herein incorporated by reference for all purposes.

A “postnatal mouse” or “transgenic postnatal mouse” refers to a mouse that has been born naturally from its mother or that is capable of survival outside the womb.

A “fragment” of a polynucleotide is a polynucleotide comprised of at least 9 contiguous nucleotides, preferably at least 15 contiguous nucleotides and more preferably at least 45 nucleotides, of coding or non-coding sequences.

The term “gene targeting” refers to a type of homologous recombination that occurs when a fragment of genomic DNA is introduced into a mammalian cell and that fragment locates and recombines with endogenous homologous sequences.

The term “homologous recombination” refers to the exchange of DNA fragments between two DNA molecules or chromatids at the site of homologous nucleotide sequences.

The term “homologous” as used herein denotes a characteristic of a DNA sequence having at least about 70 percent sequence identity as compared to a reference sequence, typically at least about 85 percent sequence identity, preferably at least about 95 percent sequence identity, and more preferably about 96, 97, 98 or 99 percent sequence identity, and most preferably about 100 percent sequence identity as compared to a reference sequence. Homology can be determined using a “BLASTN” algorithm. It is understood that homologous sequences can accommodate insertions, deletions and substitutions in the nucleotide sequence. Thus, linear sequences of nucleotides can be essentially identical even if some of the nucleotide residues do not precisely correspond or align. The reference sequence may be a subset of a larger sequence, such as a portion of a gene or flanking sequence, or a repetitive portion of a chromosome. The term “target gene” (alternatively referred to as “target gene sequence” or “target DNA sequence” or “target sequence”) refers to any nucleic acid molecule or polynucleotide of any gene to be modified by homologous recombination. The target sequence includes an intact gene, an exon or intron, a regulatory sequence or any region between genes. The target gene comprises a portion of a particular gene or genetic locus in the individual's genomic DNA. As provided herein, the target gene of the present invention is a Pten gene.

“Disruption” of a Pten gene occurs when a fragment of genomic DNA locates and recombines with an endogenous homologous sequence. These sequence disruptions or modifications may include insertions, missense, frameshift, deletion, or substitutions, or replacements of DNA sequence, or any combination thereof. Insertions include the insertion of entire genes, which may be of animal, plant, fungal, insect, prokaryotic, or viral origin. Disruption, for example, can alter or replace a promoter, enhancer, or splice site of a Pten gene, and can alter the normal gene product by inhibiting its production partially or completely or by enhancing the normal gene product's activity. Disruption can be heterozygous, i.e., affecting one chromosomal copy of the Pten gene, or homozygous, affecting both chromosomal copies of the Pten gene. Disruption of a Pten gene can inactivate the PTEN protein by removing the entire coding sequence or a fragment of the Pten coding sequence, e.g., one or more exons. In the mouse PTEN protein, exon 5 encodes a protein domain with phosphatase activity. Thus the PTEN protein can be functionally disrupted by removal of exon 5 alone, or in combination with other exons.

A “Pten-null prostate cell” is a cell or cell line from a prostate that has a homozygous Pten disruption. In preferred embodiments, the Pten-null prostate cell or cell line is derived from a transgenic mouse. Pten-null prostate cell also encompasses cells derived from metastatic carcinoma that originated from cancer of the prostate. “Prostate cell(s)” can originate from different cell types in the prostate gland and include alltypes of cells found in prostate. For example, the normal prostatic epithelium consists of at least three cell types, basal cells, secretory luminal cells and neuroendocrine cells (Bui, M. et al., Cancer Metastasis Rev 17:391-399 (1998); Isaacs, J. T. Urol Clin North Am 26:263-273 (1999)).

A “Pten nucleic acid construct” is a nucleic acid construct that can be used to disrupt a chromosomal copy of a Pten gene and typically comprises a Pten nucleic acid and specific recombination sites. The Pten nucleic acid comprises genomic Pten sequences and can include all or a portion of the Pten coding sequence. In some embodiments, the Pten nucleic acids comprises exon 5 of the Pten gene.

A “prostate-specific inducer of site-specfic recombination” is an activity that can be regulated and that can induce or initiate disruption of a Pten gene using a Pten nucleic acid construct. Examples of such inducers include proteins with recombinase activity, such as Cre or FLP recombinase. Use of Cre recombinase is described at U.S. Pat. No. 4,959,317 and use of LFP recombinase is described at U.S. Pat. No. 6,774,279, both of which are herein incorporated by reference for all purposes. In preferred embodiments, an inducer of a Pten-null nucleic acid construct is under control of a “prostate specific promoter.” A prostate specific promoter is a nucleic acid regulatory sequence that upregulates expression of an operably connected nucleic acid in prostate tissue. An example of a prostate specific promoter is the rat “probasin promoter.” See, e.g., Wu, et al., Mech. Dev. 101:61-69 (2001) and Greenberg et al., Mol. Endocrin. 8:230-239 (1994).

“Deregulated growth of prostate cells” refers to unregulated growth of prostate cells. Deregulated growth can occur in an animal or can occur in cells or cells lines derived from prostate tissue. Deregulated growth of prostate cells refers to benign and/or malignant growth of prostate cells.

“Prostate cancer” refers to a condition characterized by deregulated growth of cells in the prostate gland. Prostate cancer encompasses precancerous benign conditions that frequently lead to cancer, such as prostate cell hyperplasia, as well as recognized malignant conditions that develop during progression of prostate cancer, such as prostatic intraepithelial neoplasia, invasive adenocarcinoma of the prostate, and metastatic prostate cancer. Prostate cancer can be androgen responsive or androgen independent.

“Prostate cell hyperplasia” refers to a increase in number of prostate cells in an organ as compared to an organ from a control animal. In some embodiments prostate cell hyperplasia results in an increase in size of the prostate.

“Prostatic intraepithelial neoplasia” or “PIN” refers to the proliferation of atypical epithelial cells within pre-existing prostatic ductules and acini of the prostate. This proliferation results in stratification of the epithelial layer, giving rise to distinctive architectural features, to include cribiform, tufting or micropapillary growth patterns. Cytological atypia is characterized by nuclear enlargement, nuclear contour irregularity, hyperchromatism, prominent nucleoli accompanied with the inversion of the nuclear to cytoplasmic ratio.

“Invasive adenocarcinoma of the prostate” refers to extension of malignant cells, either as individual cells or as nests of acini, initially through the basement membrane, and subsequently the fibromuscular layer, invading into the stroma. This in turn induces both an inflammatory and a desmoplastic response. This desmoplastic response is characterized by focal stromal cellularity, found in association with the invasive cancer.

“Metastatic prostate cancer” refers to a prostate cancer cell that leaves the primary site, enters the lymphatic and blood circulatory systems, extravasates and grows as a metastatic colony. In preferred embodiments the metastatic prostate cancer cells are Pten-null cells, as described herein. Preferred metastatic sites include lymph nodes, lung, and bone.

An “effect of a composition or treatment on prostate cancer” refers to an effect of a composition or treatment on survival or proliferation of a prostate cancer cell. Generally, preferred effects decrease survival or proliferation of a prostate cancer cell. Thus, an effect includes induction of apoptosis, cell necrosis or death, and inhibition of the cell cycle in a prostate cancer cell. A composition refers to a nucleic acid or protein therapeutic agent, e.g., antisense, RNAi, antibody, or other protein or peptide. A composition also refers to a small organic molecule or a chemotherapeutic agent. A treatment includes, e.g., surgery, radiation, or heat treatment.

An “androgen independent prostate cancer cell” is a prostate cancer cell that survives in the absence of or decreased level of androgens. In some embodiments the androgen independent prostate cancer cell is able to proliferate in the absence of or decreased level of androgens.

An “androgen dependent prostate cancer cell” is a prostate cancer cell that does survive in the absence of or decreased level of androgens.

“Androgen ablation therapy” refers to a treatment or administration of a composition that decreases or eliminates the presence or effect of androgens from the body. Androgen ablation therapy can be “chemical” e.g., administration of compositions that antagonize androgen activity, such as LUPRON, ZOLADEX, FLUTAMIDE, or CASODEX. Androgen ablation therapy can be “surgical”, e.g., castration.

The term “expression pattern” or “expression profile” as used herein refers to the level of a product encoded by one or more gene(s) of interest. A product can be a nucleic acid or protein. The “expression level” as used herein refers to the amount of the product as well as the level of activity of the product. Accordingly, the expression level can be determined by measuring any number of endpoints. These endpoints include amount of mRNA, amount of protein, amount of protein activity, protein modifications, and the like.

“Biomarker” A “biomarker of prostate cancer” as used herein refers to a nucleic acid and/or protein sequence that is associated with prostate cancer. Such a biomarker is typically differentially expressed in cells derived from prostate cancer than in cells derived from a normal prostate or from an untransformed prostate cell line. Biomarkers can also be used to monitor the progression of cancer, e.g., to identify a particular stage of cancer, such as PIN, invasive adenocarcinom, or metastatic carcinoma. In addition, biomarkers can be used to identify androgen-independent prostate cancers. Biomarkers can also be used to identify precancerous conditions, such as prostate cell hyperplasia.

The term “contacting” is used herein interchangeably with the following: combined with, added to, mixed with, passed over, incubated with, flowed over, etc.

Much of the nomenclature and general laboratory procedures required in this application can be found in Sambrook, et al., Molecular Cloning: A Laboratory Manual (2nd Ed.), Vol. 1-3, Cold Spring Harbor Laboratory, Cold Spring Harbor, N.Y., 1989. The manual is hereinafter referred to as “Sambrook et al.”

The term “nucleic acid” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the complementary sequence thereof. The terms nucleic acid, “nucleic acid sequence”, and “polynucleotide” are used interchangeably herein.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (1), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor & Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that often form a compact unit of the polypeptide and are typically 25 to approximately 500 amino acids long. Typical domains are made up of sections of lesser organization such as stretches of (-sheet and (-helices. “Tertiary structure” refers to the complete three dimensional structure of a polypeptide monomer. “Quaternary structure” refers to the three dimensional structure formed, usually by the noncovalent association of independent tertiary units. Anisotropic terms are also known as energy terms.

The term “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant nucleic acid” refers to a nucleic acid that was artificially constructed (e.g., formed by linking two naturally-occurring or synthetic nucleic acid fragments). This term also applies to nucleic acids that are produced by replication or transcription of a nucleic acid that was artificially constructed. A “recombinant polypeptide” is expressed by transcription of a recombinant nucleic acid (i.e., a nucleic acid that is not native to the cell or that has been modified from its naturally occurring form), followed by translation of the resulting transcript.

A “heterologous polynucleotide”, “heterologous nucleic acid”, “heterologous polypeptide” or “heterologous protein” as used herein, is one that originates from a source foreign to the particular host cell, or, if from the same source, is modified from its original form. Thus, a heterologous nucleic acid in a prokaryotic host cell includes a nucleic acid that is endogenous to the particular host cell but has been modified. Modification of the heterologous sequence may occur, e.g., by treating the DNA with a restriction enzyme to generate a DNA fragment that is capable of being operably linked to a promoter. Techniques such as site-directed mutagenesis are also useful for modifying a heterologous sequence.

A “subsequence” refers to a sequence of nucleic acids or amino acids that comprise a part of a longer sequence of nucleic acids or amino acids (e.g., polypeptide) respectively.

A “recombinant expression cassette” or simply an “expression cassette” is a nucleic acid construct, generated recombinantly or synthetically, with nucleic acid elements that are capable of affecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

The term “isolated” refers to material that is substantially or essentially free from components which interfere with the activity of an enzyme. For cells, saccharides, nucleic acids, and polypeptides of the invention, the term “isolated” refers to material that is substantially or essentially free from components which normally accompany the material as found in its native state. Typically, isolated saccharides, proteins or nucleic acids of the invention are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligonucleotides, or other sialylated products, purity can be determined using, e.g., thin layer chromatography, HPLC, or mass spectroscopy.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least 60%, preferably 80% or 85%, most preferably at least 90%, 91%, 92%, 93%, 94%, 95%, 96%, 97%, 98%, or 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na⁺ ion, typically about 0.01 to 1.0 M Na⁺ ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90-95° C. for 30-120 sec, an annealing phase lasting 30-120 sec, and an extension phase of about 72° C. for 1-2 min. Protocols and guidelines for low and high stringency amplification reactions are available, e.g., in Innis, et al. (1990) PCR Protocols: A Guide to Methods and Applications Academic Press, N.Y.

The phrases “specifically binds to” or “specifically immunoreactive with”, when referring to an antibody refers to a binding reaction which is determinative of the presence of the protein or other antigen in the presence of a heterogeneous population of proteins, saccharides, and other biologics. Thus, under designated immunoassay conditions, the specified antibodies bind preferentially to a particular antigen and do not bind in a significant amount to other molecules present in the sample. Specific binding to an antigen under such conditions requires an antibody that is selected for its specificity for a particular antigen. A variety of immunoassay formats can be used to select antibodies specifically immunoreactive with a particular antigen. For example, solid-phase ELISA immunoassays are routinely used to select monoclonal antibodies specifically immunoreactive with an antigen. See Harlow and Lane (1988) Antibodies, A Laboratory Manual, Cold Spring Harbor Publications, New York, for a description of immunoassay formats and conditions that can be used to determine specific immunoreactivity.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F (ab)′₂, a dimer of Fab which itself is a light chain joined to V_{H}-C_{H}1 by a disulfide bond. The F (ab)′₂ may be reduced under mild conditions to break the disulfide linkage in the hinge region, thereby converting the F (ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

In one embodiment, the antibody is conjugated to an “effector” moiety. The effector moiety can be any number of molecules, including labeling moieties such as radioactive labels or fluorescent labels for use in diagnostic assays.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to IgE protein, polymorphic variants, alleles, orthologs, and conservatively modified variants, or splice variants, or portions thereof, can be selected to obtain only those polyclonal antibodies that are specifically immunoreactive with IgE proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

An “antigen” is a molecule that is recognized and bound by an antibody, e.g., peptides, carbohydrates, organic molecules, or more complex molecules such as glycolipids and glycoproteins. The part of the antigen that is the target of antibody binding is an antigenic determinant and a small functional group that corresponds to a single antigenic determinant is called a hapten.

A “label” is a composition detectable by spectroscopic, photochemical, biochemical, immunochemical, or chemical means. For example, useful labels include ³²P, ¹²⁵I, fluorescent dyes, electron-dense reagents, enzymes (e.g., as commonly used in an ELISA), biotin, digoxigenin, or haptens and proteins for which antisera or monoclonal antibodies are available (e.g., the polypeptide of SEQ ID NO:3 can be made detectable, e.g., by incorporating a radiolabel into the peptide, and used to detect antibodies specifically reactive with the peptide).

The term “immunoassay” is an assay that uses an antibody to specifically bind an antigen. The immunoassay is characterized by the use of specific binding properties of a particular antibody to isolate, target, and/or quantify the antigen.

The term “carrier molecule” means an immunogenic molecule containing antigenic determinants recognized by T cells. A carrier molecule can be a protein or can be a lipid. A carrier protein is conjugated to a polypeptide to render the polypeptide immunogenic. Carrier proteins include keyhole limpet hemocyanin, horseshoe crab hemocyanin, and bovine serum albumin.

The term “adjuvant” means a substance that nonspecifically enhances the immune response to an antigen. Adjuvants include Freund's adjuvant, either complete or incomplete; Titermax gold adjuvant; alum; and bacterial LPS.

“Biological sample” as used herein is a sample of biological tissue or fluid that contains nucleic acids or polypeptides. Such samples include, but are not limited to, tissue isolated from primates, e.g., humans, or rodents, e.g., mice, and rats. Biological samples may also include sections of tissues such as biopsy and autopsy samples, frozen sections taken for histologic purposes, blood, plasma, serum, sputum, stool, tears, mucus, hair, skin, bone cartilage, etc. Biological samples also include explants and primary and/or transformed cell cultures derived from patient tissues. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

“Providing a biological sample” means to obtain a biological sample for use in methods described in this invention. Most often, this will be done by removing a sample of cells from an animal, but can also be accomplished by using previously isolated cells (e.g., isolated by another person, at another time, and/or for another purpose), or by performing the methods of the invention in vivo. Archival tissues, having treatment or outcome history, will be particularly useful.

III. Pten Nucleic Acids and Proteins

A “Pten gene” refers to a nucleic acid that encodes a PTEN protein. In preferred embodiments, a Pten gene is a mouse Pten gene. The mouse PTEN protein sequence is found at Accession number O08586 and the encoding nucleic acid sequence is found at Accession number NM_{—}008960, each of which are herein incorporated by reference. The mouse Pten gene has been mapped to mouse chromosome 19 and the locus tag is MGI:109583, which is also herein incorporated by reference for all purposes.

The PTEN protein has phosphatase activity and antagonizes the PI3K/AKT pathway (Cantley, L. C. et al., Proc Natl Acad Sci USA 96:4240-4245 (1999); Di Cristofano, A. et al., Cell 100:387-390 (2000); Meehama, T. et al., Annu. Rev. Biochem. 70:247-279 (2001)). Loss of PTEN function, either in murine embryonic stem cells or in human cancer cell lines, results in accumulation of PIP3 and activation of its downstream effectors, such as AKT/PKB Stambolic, V., et al. Cell 95:29-39 (1998); Sun, H. et al., Proc. Natl. Acad. Sci. USA 96 96:6199-6204 (1999); Wu, X. et al., Proc. Natl. Acad. Sci. USA 95:15587-15591 (1998)). As a serine/threonine protein kinase, AKT functions by phosphorylating key intermediate signaling molecules, such as glycogen synthase kinase-3 (GSK3), BAD, Caspase 9, IκB, leading to increased cell metabolism, cell growth, and cell survival (Di Cristofano, A. et al., Cell 100:387-390 (2000); Hanahan, D. et al., Cell 100:57-70 (2000); Vivanco, I. et al., Nat. Rev. Cancer 2:489-501 (2002)). One indication of PTEN function is a change in the ratio of phosphorylated AKT to unphosphorylated AKT. In the presence of functional PTEN protein, more AKT protein is unphosphorylated.

PTEN function can be determined in a variety of ways. For example, levels of nucleic acid that encode PTEN protein can be determined. In addition, where PTEN nucleic acids have been disrupted, such disruptions can be detected using, e.g., hybridization assays or PCR to identify a disruption in a PTEN nucleic acid. PTEN function can also be determined by assaying the presence or amount of PTEN protein, typically by immunological methods. In addition, PTEN function can be determined by assaying PTEN activity or the activity of a biochemical pathway that is regulated by the PTEN protein, e.g., the PIP3, AKT/PKB pathway.

VI. Pten-Null Cells and Animals

The animals, cells, and methods of the invention are preformed using Pten-null cells and animals. Pten-null cells and animals are generated as described herein, typically by targeting a genomic copy of the Pten gene for disruption and ultimately by eliminating or greatly decreasing PTEN function in an animal or cell. Preferably, such targeted disruption will occur in the prostate of the animal. In a more preferred embodiment, Pten gene disruption will occur almost exclusively or exclusively in prostate tissue.

Generation of Targeting Construct

The targeting construct of the present invention may be produced using standard methods known in the art. (see, e.g., Sambrook, et al., 1989, Molecular Cloning: A Laboratory Manual, Second Edition, Cold Spring Harbor Laboratory Press, Cold Spring Harbor, N.Y.; E. N. Glover (eds.), 1985, DNA Cloning: A Practical Approach, Volumes I and II; M. J. Gait (ed.), 1984, Oligonucleotide Synthesis; B. D. Hames & S. J. Higgins (eds.), 1985, Nucleic Acid Hybridization; B. D. Hames & S. J. Higgins (eds.), 1984, Transcription and Translation; R. I. Freshney (ed.), 1986, Animal Cell Culture; Immobilized Cells and Enzymes, IRL Press, 1986; B. Perbal, 1984, A Practical Guide To Molecular Cloning; F. M. Ausubel et al., 1994, Current Protocols in Molecular Biology, John Wiley & Sons, Inc.). For example, the targeting construct may be prepared in accordance with conventional ways, where sequences may be synthesized, isolated from natural sources, manipulated, cloned, ligated, subjected to in vitro mutagenesis, primer repair, or the like. At various stages, the joined sequences may be cloned, and analyzed by restriction analysis, sequencing, or the like.

The targeting DNA can be constructed using techniques well known in the art. For example, the targeting DNA may be produced by chemical synthesis of oligonucleotides, nick-translation of a double-stranded DNA template, polymerase chainreaction amplification of a sequence (or ligase chain reaction amplification), purification of prokaryotic or target cloning vectors harboring a sequence of interest (e.g., a cloned cDNA or genomic DNA, synthetic DNA or from any of the aforementioned combination) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence. Moreover, the length of homology may be selected using known methods in the art. For example, selection may be based on the sequence composition and complexity of the predetermined endogenous target DNA sequence(s).

The targeting construct of the present invention typically comprises a first sequence homologous to a portion or region of the Pten gene and a second sequence homologous to a second portion or region of the Pten gene. The targeting construct further comprises a positive selection marker, which is preferably positioned in between the first and the second DNA sequence that are homologous to a portion or region of the target DNA sequence. The positive selection marker may be operatively linked to a promoter and a polyadenylation signal.

Other regulatory sequences known in the art may be incorporated into the targeting construct to disrupt or control expression of a particular gene in a specific cell type. In addition, the targeting construct may also include a sequence coding for a screening marker, for example, green fluorescent protein (GFP), or another modified fluorescent protein.

Although the size of the homologous sequence is not critical and can range from as few as 50 base pairs to as many as 100 kb, preferably each fragment is greater than about 1 kb in length, more preferably between about 1 and about 10 kb, and even more preferably between about 1 and about 5 kb. One of skill in the art will recognize that although larger fragments may increase the number of homologous recombination events in ES cells, larger fragments will also be more difficult to clone.

Generally, a sequence of interest is identified and isolated from a plasmid library in a single step using, for example, long-range PCR. Following isolation of this sequence, a second polynucleotide that will disrupt the target sequence can be readily inserted between two regions encoding the sequence of interest. In accordance with this aspect, the construct is generated in two steps by (1) amplifying (for example, using long-range PCR) sequences homologous to the target sequence, and (2) inserting another polynucleotide (for example a selectable marker) into the PCR product so that it is flanked by the homologous sequences. Typically, the vector is a plasmid from a plasmid genomic library. The completed construct is also typically a circular plasmid.

In another embodiment, the targeting construct may contain more than one selectable maker gene, including a negative selectable marker, such as the herpes simplex virus tk (HSV-tk) gene. The negative selectable marker may be operatively linked to a promoter and a polyadenylation signal. (see, e.g., U.S. Pat. No. 5,464,764; U.S. Pat. No. 5,487,992; U.S. Pat. No. 5,627,059; and U.S. Pat. No. 5,631,153).

Generation of Cells and Confirmation of Homologous Recombination Events

Once an appropriate targeting construct has been prepared, the targeting construct may be introduced into an appropriate host cell using any method known in the art. Various techniques may be employed in the present invention, including, for example, pronuclear microinjection; retrovirus mediated gene transfer into germ lines; gene targeting in embryonic stem cells; electroporation of embryos; sperm-mediated gene transfer; and calcium phosphate/DNA co-precipitates, microinjection of DNA into the nucleus, bacterial protoplast fusion with intact cells, transfection, polycations, e.g., polybrene, polyomithine, etc., or the like (see, e.g., U.S. Pat. No. 4,873,191; Van der Putten, et al., 1985, Proc. Natl. Acad. Sci., USA 82:6148-6152; Thompson, et al., 1989, Cell 56:313-321; Lo, 1983, Mol Cell. Biol. 3:1803-1814; Lavitrano, et al., 1989, Cell, 57:717-723). Various techniques for transforming mammalian cells are known in the art. (see, e.g., Gordon, 1989, Intl. Rev. Cytol., 115:171-229; Keown et al., 1989, Methods in Enzymology; Keown et al., 1990, Methods and Enzymology, Vol. 185, pp. 527-537; Mansour et al., 1988, Nature, 336:348-352).

In a preferred aspect of the present invention, the targeting construct is introduced into host cells by electroporation. In this process, electrical impulses of high field strength reversibly permeabilize biomembranes allowing the introduction of the construct. The pores created during electroporation permit the uptake of macromolecules such as DNA. (see, e.g., Potter, H., et al., 1984, Proc. Nat'l. Acad. Sci. U.S.A. 81:7161-7165).

Any cell type capable of homologous recombination may be used in the practice of the present invention. Examples of such target cells include cells derived from vertebrates including mammals such as humans, bovine species, ovine species, murine species, simian species, and ether eucaryotic organisms such as filamentous fungi, and higher multicellular organisms such as plants.

Preferred cell types include embryonic stem (ES) cells, which are typically obtained from pre-implantation embryos cultured in vitro. (see, e.g., Evans, M. J., et al., 1981, Nature 292:154-156; Bradley, M. O., et al., 1984, Nature 309:255-258; Gossler et al., 1986, Proc. Natl. Acad. Sci. USA 83:9065-9069; and Robertson, et al., 1986, Nature 322:445-448). The ES cells are cultured and prepared for introduction of the targeting construct using methods well known to the skilled artisan. (see, e.g., Robertson, E. J. ed. “Teratocarcinomas and Embryonic Stem Cells, a Practical Approach”, IRL Press, Washington D.C., 1987; Bradley et al., 1986, Current Topics in Devel. Biol. 20:357-371; by Hogan et al., in “Manipulating the Mouse Embryo”: A Laboratory Manual, Cold Spring Harbor Laboratory Press, Cold Spring Harbor N.Y., 1986; Thomas et al., 1987, Cell 51:503; Koller et al., 1991, Proc. Natl. Acad. Sci. USA, 88:10730; Dorinetal., 1992, Transgenic Res. 1:101; and Veis et al., 1993, Cell 75:229). The ES cells that will be inserted with the targeting construct are derived from an embryo or blastocyst of the same species as the developing embryo into which they are to be introduced. ES cells are typically selected for their ability to integrate into the inner cell mass and contribute to the germ line of an individual when introduced into the mammal in an embryo at the blastocyst stage of development. Thus, any ES cell line having this capability is suitable for use in the practice of the present invention.

The present invention may also be used to knockout genes in other cell types, such as stem cells. By way of example, stem cells may be myeloid, lymphoid, or neural progenitor and precursor cells. These cells comprising a disruption or knockout of a gene may be particularly useful in the study of Pten gene function in individual developmental pathways. Stem cells may be derived from any vertebrate species, such as mouse, rat, dog, cat, pig, rabbit, human, non-human primates and the like.

After the targeting construct has been introduced into cells, the cells where successful gene targeting has occurred are identified. Insertion of the targeting construct into the targeted gene is typically detected by identifying cells for expression of the marker gene. In a preferred embodiment, the cells transformed with the targeting construct of the present invention are subjected to treatment with an appropriate agent that selects against cells not expressing the selectable marker. Only those cells expressing the selectable marker gene survive and/or grow under certain conditions. For example, cells that express the introduced neomycin resistance gene are resistant to the compound G418, while cells that do not express the neo gene marker are killed by G418. If the targeting construct also comprises a screening marker such as GFP, homologous recombination can be identified through screening cell colonies under a fluorescent light. Cells that have undergone homologous recombination will have deleted the GFP gene and will not fluoresce.

If a regulated positive selection method is used in identifying homologous recombination events, the targeting construct is designed so that the expression of the selectable marker gene is regulated in a manner such that expression is inhibited following random integration but is permitted (derepressed) following homologous recombination. More particularly, the transfected cells are screened for expression of the neo gene, which requires that (1) the cell was successfully electroporated, and (2) lac repressor inhibition of neo transcription was relieved by homologous recombination. This method allows for the identification of transfected cells and homologous recombinants to occur in one step with the addition of a single drug.

Alternatively, a positive-negative selection technique may be used to select homologous recombinants. This technique involves a process in which a first drug is added to the cell population, for example, a neomycin-like drug to select for growth of transfected cells, i.e. positive selection. A second drug, such as FIAU is subsequently added to kill cells that express the negative selection marker, i.e. negative selection. Cells that contain and express the negative selection marker are killed by a selecting agent, whereas cells that do not contain and express the negative selection marker survive. For example, cells with non-homologous insertion of the construct express HSV thymidine kinase and therefore are sensitive to the herpes drugs such as gancyclovir (GANC) or FIAU (1-(2-deoxy 2-fluoro-B-D-arabinofluranosyl)-5-iodouracil). (see, e.g., Mansour et al., Nature 336:348-352: (1988); Capecchi, Science 244:1288-1292, (1989); Capecchi, Trends in Genet. 5:70-76 (1989)).

Successful recombination may be identified by analyzing the DNA of the selected cells to confirm homologous recombination. Various techniques known in the art, such as PCR and/or Southern analysis may be used to confirm homologous recombination events.

Homologous recombination may also be used to disrupt genes in stem cells, and other cell types, which are not totipotent embryonic stem cells. By way of example, stem cells may be myeloid, lymphoid, or neural progenitor and precursor cells. Such transgenic cells may be particularly useful in the study of Pten gene function in individual developmental pathways. Stem cells may be derived from any vertebrate species, such as mouse, rat, dog, cat, pig, rabbit, human, non-human primates and the like.

In cells that are not totipotent it may be desirable to knock out both copies of the target using methods that are known in the art. For example, cells comprising homologous recombination at a target locus that have been selected for expression of a positive selection marker (e.g., Neo^{r}) and screened for non-random integration, can be further selected for multiple copies of the selectable marker gene by exposure to elevated levels of the selective agent (e.g., G418). The cells are then analyzed for homozygosity at the target locus. Alternatively, a second construct can be generated with a different positive selection marker inserted between the two homologous sequences. The two constructs can be introduced into the cell either sequentially or simultaneously, followed by appropriate selection for each of the positive marker genes. The final cell is screened for homologous recombination of both alleles of the target.

Production of Transgenic Animals

Selected cells are then injected into a blastocyst (or other stage of development suitable for the purposes of creating a viable animal, such as, for example, a morula) of an animal (e.g., a mouse) to form chimeras (see e.g., Bradley, A. in Teratocarcinomas and Embryonic Stem Cells: A Practical Approach, E. J. Robertson, ed., IRL, Oxford, pp. 113-152 (1987)). Alternatively, selected ES cells can be allowed to aggregate with dissociated mouse embryo cells to form the aggregation chimera. A chimeric embryo can then be implanted into a suitable pseudopregnant female foster animal and the embryo brought to term. Chimeric progeny harbouring the homologously recombined DNA in their germ cells can be used to breed animals in which all cells of the animal contain the homologously recombined DNA. In one embodiment, chimeric progeny mice are used to generate a mouse with a heterozygous disruption in the Pten gene. Heterozygous transgenic mice can then be mated. It is well know in the art that typically ¼ of the offspring of such matings will have a homozygous disruption in the Pten gene.

The heterozygous and homozygous transgenic mice can then be compared to normal, wild type mice to determine whether disruption of the Pten gene causes phenotypic changes, especially pathological changes. For example, heterozygous and homozygous mice may be evaluated for phenotypic changes by physical examination, necropsy, histology, clinical chemistry, complete blood count, body weight, organ weights, and cytological evaluation of e.g., prostate tissue and bone marrow.

In one embodiment, the phenotype (or phenotypic change) associated with a disruption in the Pten gene is placed into or stored in a database. Preferably, the database includes: (i) genotypic data (e.g., identification of the disrupted gene) and (ii) phenotypic data (e.g., phenotype(s) resulting from the gene disruption) associated with the genotypic data. The database is preferably electronic. In addition, the database is preferably combined with a search tool so that the database is searchable.

Conditional Transgenic Animals

The present invention further contemplates conditional transgenic or knockout animals, such as those produced using recombination methods. Bacteriophage P1 Cre recombinase and flp recombinase from yeast plasmids are two non-limiting examples of site-specific DNA recombinase enzymes that cleave DNA at specific target sites (lox P sites for cre recombinase and frt sites for flp recombinase) and catalyze a ligation of this DNA to a second cleaved site. A large number of suitable alternative site-specific recombinases have been described, and their genes can be used in accordance with the method of the present invention. Such recombinases include the Int recombinase of bacteriophage lambda. (with or without X is) (Weisberg, R. et al., in Lambda II, (Hendrix, R., et al., Eds.), Cold Spring Harbor Press, Cold Spring Harbor, N.Y., pp. 211-50 (1983), herein incorporated by reference); TpnI and the .beta.-lactamase transposons (Mercier, et al., J. Bacteriol., 172:3745-57 (1990)); the Tn3 resolvase (Flanagan & Fennewald J. Molec. Biol., 206:295-304 (1989); Stark, et al., Cell, 58:779-90 (1989)); the yeast recombinases (Matsuzaki, et al., J. Bacteriol., 172:610-18 (1990)); the B. subtilis SpoIVC recombinase (Sato, et al., J. Bacteriol. 172:1092-98 (1990)); the Flp recombinase (Schwartz & Sadowski, J. Molec. Biol., 205:647-658 (1989); Parsons, et al., J. Biol. Chem., 265:4527-33 (1990); Golic & Lindquist, Cell, 59:499-509 (1989); Amin, et al., J. Molec. Biol., 214:55-72 (1990)); the Hin recombinase (Glasgow, et al., J. Biol. Chem., 264:10072-82 (1989)); immunoglobulin recombinases (Malynn, et al., Cell, 54:453-460 (1988)); and the Cin recombinase (Haffter & Bickle, EMBO J., 7:3991-3996 (1988); Hubner, et al., J. Molec. Biol., 205:493-500 (1989)), all herein incorporated by reference. Such systems are discussed by Echols (J. Biol. Chem. 265:14697-14700 (1990)); de Villartay (Nature, 335:170-74 (1988)); Craig, (Ann. Rev. Genet., 22:77-105 (1988)); Poyart-Salmeron, et al., (EMBO J. 8:2425-33 (1989)); Hunger-Bertling, et al., (Mol Cell. Biochem., 92:107-16 (1990)); and Cregg & Madden (Mol. Gen. Genet., 219:320-23 (1989)), all herein incorporated by reference.

Cre has been purified to homogeneity, and its reaction with the loxP site has been extensively characterized (Abremski & Hess J. Mol. Biol. 259:1509-14 (1984), herein incorporated by reference). Cre protein has a molecular weight of 35,000 and can be obtained commercially from New England Nuclear/Du Pont. The cre gene (which encodes the Cre protein) has been cloned and expressed (Abremski, et al., Cell 32:1301-11 (1983), herein incorporated by reference). The Cre protein mediates recombination between two loxP sequences (Sternberg, et al., Cold Spring Harbor Symp. Quant. Biol. 45:297-309 (1981)), which may be present on the same or different DNA molecule. Because the internal spacer sequence of the loxP site is asymmetrical, two loxP sites can exhibit directionality relative to one another (Hoess & Abremski Proc. Natl. Acad. Sci. U.S.A. 81:1026-29 (1984)). Thus, when two sites on the same DNA molecule are in a directly repeated orientation, Cre will excise the DNA between the sites (Abremski, et al., Cell 32:1301-11 (1983)). However, if the sites are inverted with respect to each other, the DNA between them is not excised after recombination but is simply inverted. Thus, a circular DNA molecule having two loxP sites in direct orientation will recombine to produce two smaller circles, whereas circular molecules having two loxP sites in an inverted orientation simply invert the DNA sequences flanked by the loxP sites. In addition, recombinase action can result in reciprocal exchange of regions distal to the target site when targets are present on separate DNA molecules.

Recombinases have important application for characterizing gene function in knockout models. When the constructs described herein are used to disrupt Pten genes, a fusion transcript can be produced when insertion of the positive selection marker occurs downstream (3′) of the translation initiation site of the Pten gene. The fusion transcript could result in some level of protein expression with unknown consequence. It has been suggested that insertion of a positive selection marker gene can affect the expression of nearby genes. These effects may make it difficult to determine gene function after a knockout event since one could not discern whether a given phenotype is associated with the inactivation of a gene, or the transcription of nearby genes. Both potential problems are solved by exploiting recombinase activity. When the positive selection marker is flanked by recombinase sites in the same orientation, the addition of the corresponding recombinase will result in the removal of the positive selection marker. In this way, effects caused by the positive selection marker or expression of fusion transcripts are avoided.

In one embodiment, purified recombinase enzyme is provided to the cell by direct microinjection. In another embodiment, recombinase is expressed from a co-transfected construct or vector in which the recombinase gene is operably linked to a functional promoter. An additional aspect of this embodiment is the use of tissue-specific or inducible recombinase constructs that allow the choice of when and where recombination occurs. One method for practicing the inducible forms of recombinase-mediated recombination involves the use of vectors that use inducible or tissue-specific promoters or other gene regulatory elements to express the desired recombinase activity. The inducible expression elements are preferably operatively positioned to allow the inducible control or activation of expression of the desired recombinase activity. Examples of such inducible promoters or other gene regulatory elements include, but are not limited to, tetracycline, metallothionine, ecdysone, and other steroid-responsive promoters, rapamycin responsive promoters, and the like (No, et al., Proc. Natl. Acad. Sci. USA, 93:3346-51 (1996); Furth, et al., Proc. Natl. Acad. Sci. USA, 91:9302-6 (1994)). Additional control elements that can be used include promoters requiring specific transcription factors such as viral, promoters. Vectors incorporating such promoters would only express recombinase activity in cells that express the necessary transcription factors.

V. Models of Prostate Cancer and Prostate Cell Growth

The present invention provides models for analysis of prostate cancer progression or of disregulation of prostate cell proliferation in a mammal, e.g., a mouse. In preferred embodiments, prostate cancer progression or disregulation of prostate cell proliferation are analyzed in a male animal. In most preferred embodiments, prostate cancer progression or disregulation of prostate cell proliferation are analyzed in a postnatal animal.

Homozygous disruption of the mouse Pten gene in the prostate results in prostate hyperplasia followed by regular progression from PIN to invasive carcinoma to metastatic carcinoma. This disease progression closely follows prostate cancer progression in humans. Animals comprising a homozygous disruption of the mouse Pten gene can be used to analyze prostate cancer progression. In addition, cancerous cells can be obtained from the Pten-null animals and used for analysis of the molecular basis of the disease.

Homozygous disruption of the mouse Pten gene in the prostate can also result in androgen independent prostate cancer. Androgen independent cancer cells are characterized by their ability to survive after treatment with androgen ablation therapy.

Because of the similarity in progression between human prostate cancer and the murine cancer related to prostate specific Pten disruption, murine Pten-related prostate cancer can be used to identify compounds and treatments that have a therapeutic effect on human prostate cancer. Compounds or treatments can be tested on whole animals, i.e., mice, that have a prostate specific Pten disruption or can be tested on cells or cell lines derived from animals that have a prostate specific Pten disruption. In addition, androgen independent murine Pten-related prostate cancer can be used to identify compounds and treatments that have a therapeutic effect on androgen independent human prostate cancer.

VI. Compositions or Treatments that Affect Pten-Related Prostate Cancer

Compositions or treatments that affect Pten-related prostate cancer or disregulated cell growth can be identified using the prostate specific Pten disruption animals described herein, or using cells or cell lines isolated from such animals. Assays to determine an effect of a composition or treatment on Pten-related prostate cancer or disregulated cell growth are described herein and can be performed on androgen dependent or androgen independent prostate cancer cells or on whole animals that comprise such cells.

Compounds that affect the Pten signaling pathway can effect Pten-related prostate cancer or disregulated cell growth. These compounds can be tested for therapeutic effect on prostate cancer in combination with compounds that affect other signaling pathways that are deregulated in prostate cancer, e.g. the p53 signaling pathway, the wnt/fzd signaling pathway, and the BMP signaling pathway.

Assays Using Whole Animals

A compound or treatment can be assayed for an effect on a Pten-related prostate cancer by administering the compound or treatment to an animal that has a prostate specific disruption of the Pten gene and also has or is suspected of having a Pten-related prostate cancer or Pten-related disregulated cell growth.

Compounds or treatments that have an effect on a Pten-related prostate cancer or Pten-related disregulated cell growth can inhibit growth or proliferation of Pten-related prostate cancer cells or Pten-related disregulated cell growth. The compounds or treatments are preferably also performed on a control animal that does not have a prostate specific disruption of the Pten gene and the effect of the compound is seen by comparison of the prostate specific Pten disrupted animal to the control animal. Another control is an untreated animal that does have a prostate specific disruption of the Pten gene.

Effect of a compound or treatment on a whole animal include increased lifespan, failure to progress from one prostate cancer stage to another, e.g., failure to progress from PIN to invasive carcinoma, or failure to progress from invasive carcinoma to metastatic carcinoma. Other effects include decreased proliferation of cancer cells in the animal, e.g., as assayed by Ki567 staining, or by increase apoptosis of cancer cells, e.g., as assayed by TUNEL staining.

Assays Using Prostate Cells with Pten Disruptions

Compounds that affect a Pten-related prostate cancer or Pten-related disregulated cell growth will likely have an effect on growth or proliferation of prostate cancer cells derived from prostate tissue that has a Pten disruption. Changes in cell growth can be assessed by using a variety of in vitro and in vivo assays, e.g., changes in apoptosis, changes in cell cycle pattern, etc. The prostate cancer cells derived from prostate tissue that has a Pten disruption are grown in an appropriate medium and contacted with test compound to assess its effect on Pten-related prostate cancer or Pten-related disregulated cell growth.

Apoptosis Analysis

Apoptosis analysis can be used as an assay to identify compounds that affect a Pten-related prostate cancer or Pten-related disregulated cell growth. The apoptotic change can be determined using methods known in the art, such as DAPI staining and TUNEL assay using fluorescent microscope. For TUNEL assay, commercially available kit can be used (e.g., Fluorescein FragEL DNA Fragmentation Detection Kit (Oncogene Research Products, Cat.# QIA39)+Tetramethyl-rhodamine-5-dUTP (Roche, Cat. # 1534 378)). G₀/G₁ Cell Cycle Arrest Analysis

G₀/G₁ cell cycle arrest can be used as an assay to identify compounds that affect a Pten-related prostate cancer or Pten-related disregulated cell growth. Compounds that inhibit cancer cell growth can cause G₀/G₁ cell cycle arrest. Methods known in the art can be used to measure the degree of G₁ cell cycle arrest. For example, the propidium iodide signal can be used as a measure for DNA content to determine cell cycle profiles on a flow cytometer. The percent of the cells in each cell cycle can be calculated.

Modulators that Affect Pten-Related Prostate Cancer

The compounds tested as modulators or as having an effect on Pten-related prostate cancer or disregulated cell growth can be any small chemical compound, or a biological entity, such as a protein, sugar, nucleic acid or lipid.

Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Solid State and Soluble High Throughput Assays

In one embodiment the invention provide soluble assays a cell or tissue with Pten-related prostate cancer or Pten-related disregulated cell growth. In another embodiment, the invention provides solid phase based in vitro assays in a high throughput format, where the cell or tissue with Pten-related prostate cancer or Pten-related disregulated cell growth.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-1500 different compounds. It is possible to assay several different plates per day; assay screens for up to about 6,000-20,000 different compounds is possible using the integrated systems of the invention.

The molecule of interest can be bound to the solid state component, directly or indirectly, via covalent or non covalent linkage, e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

VII. Analysis of Expression Patterns and Identification of Pten-Related Prostate Cancer Biomarkers

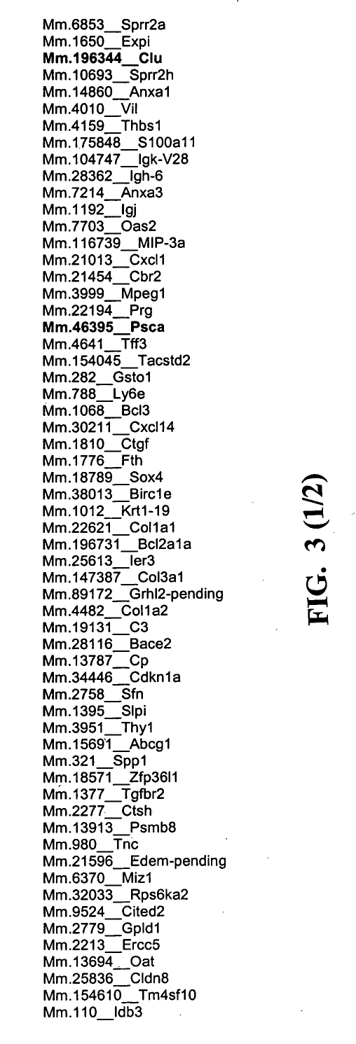

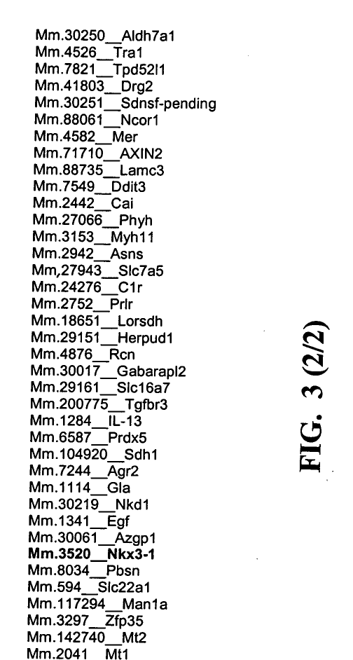

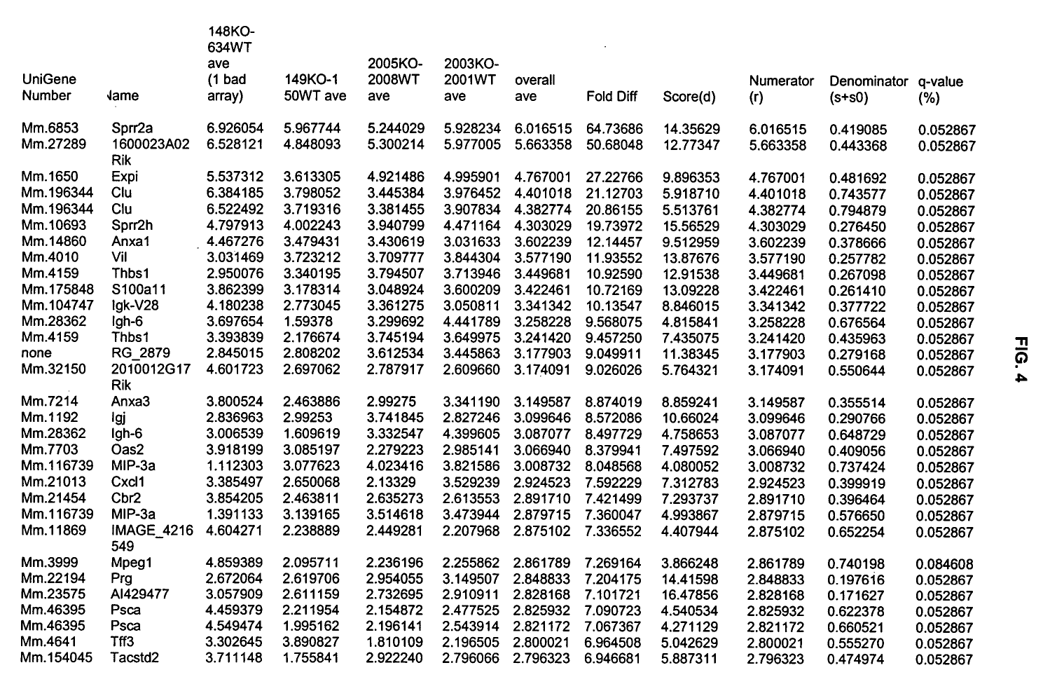

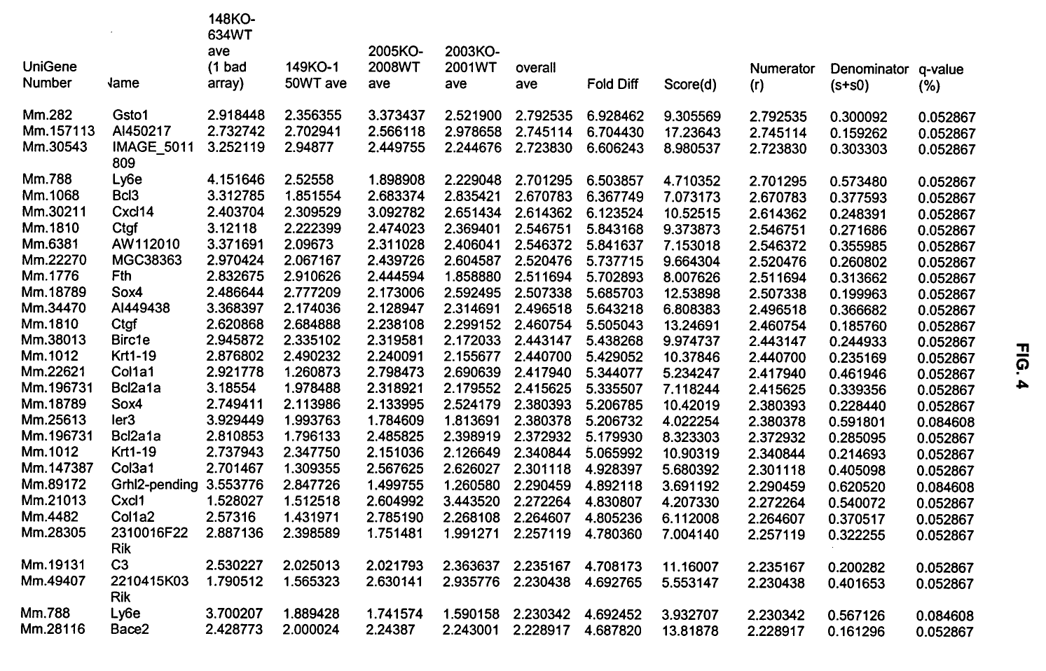

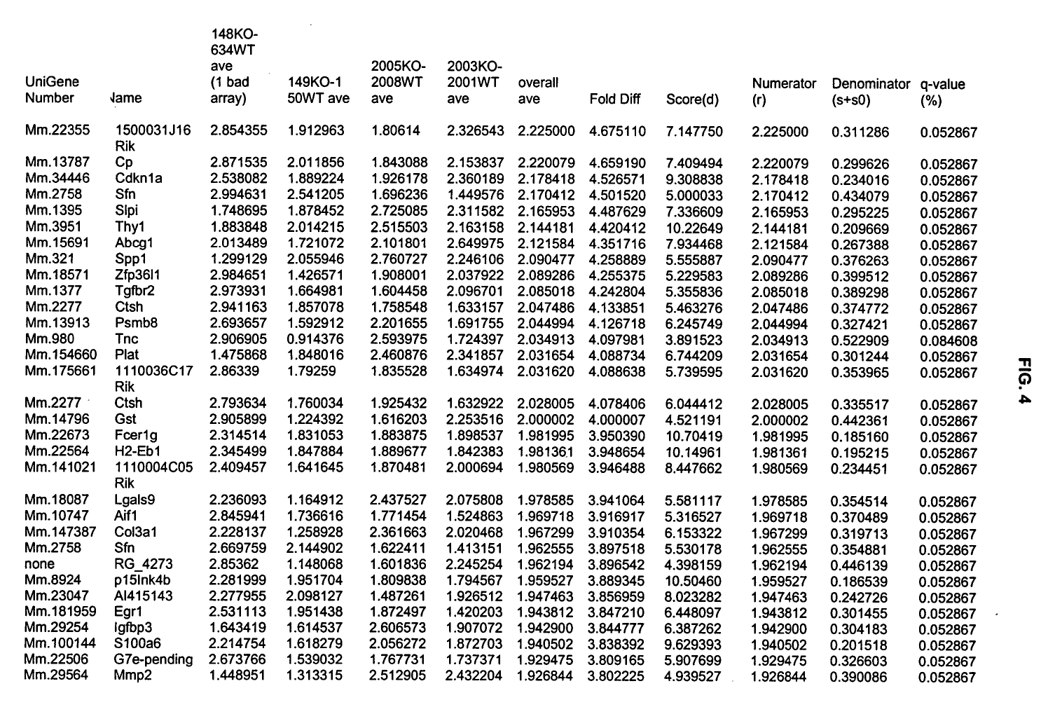

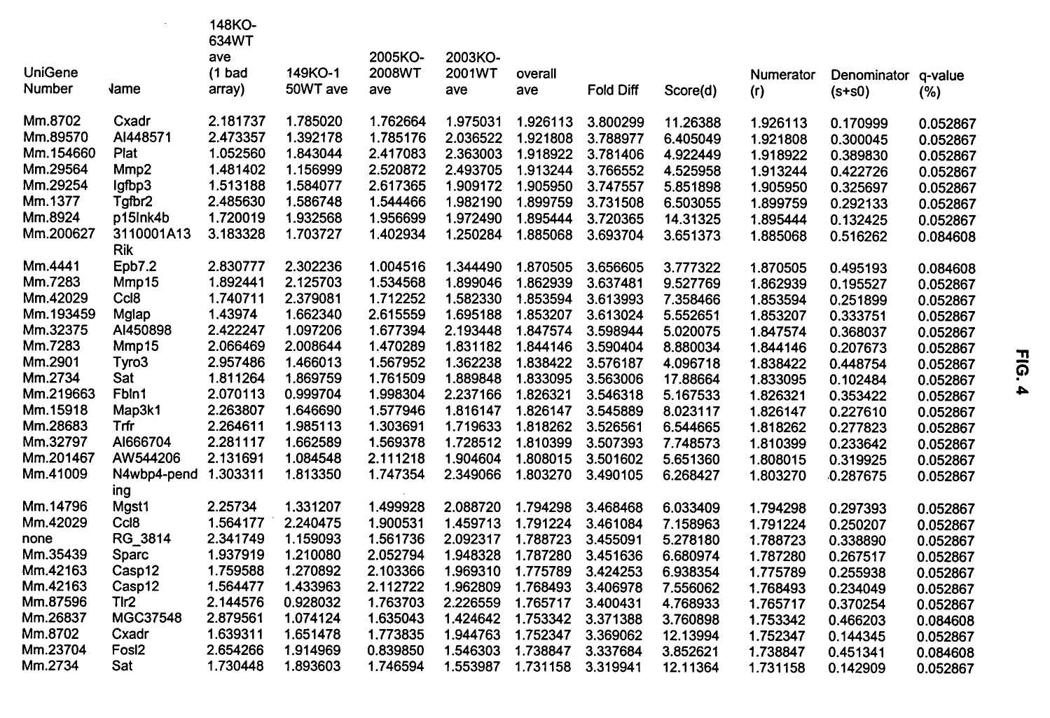

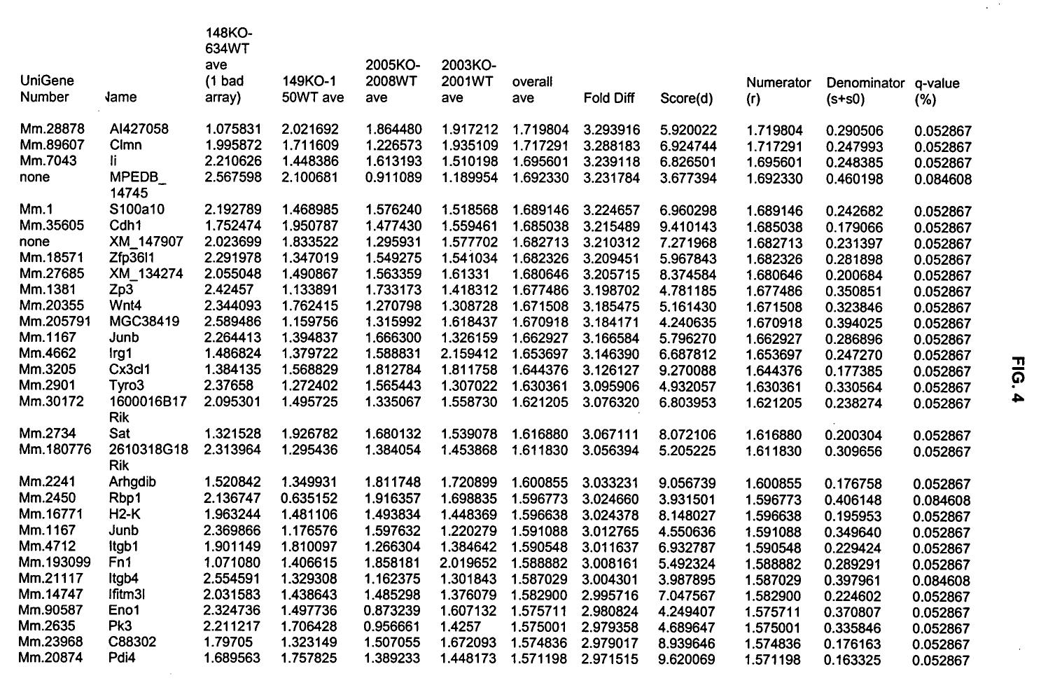

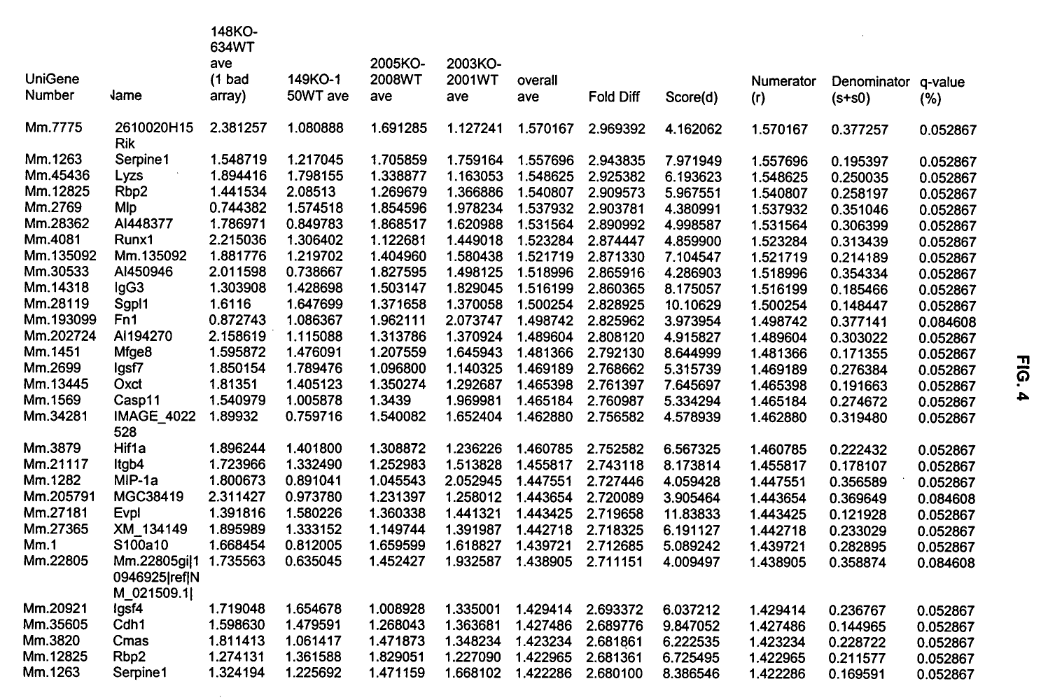

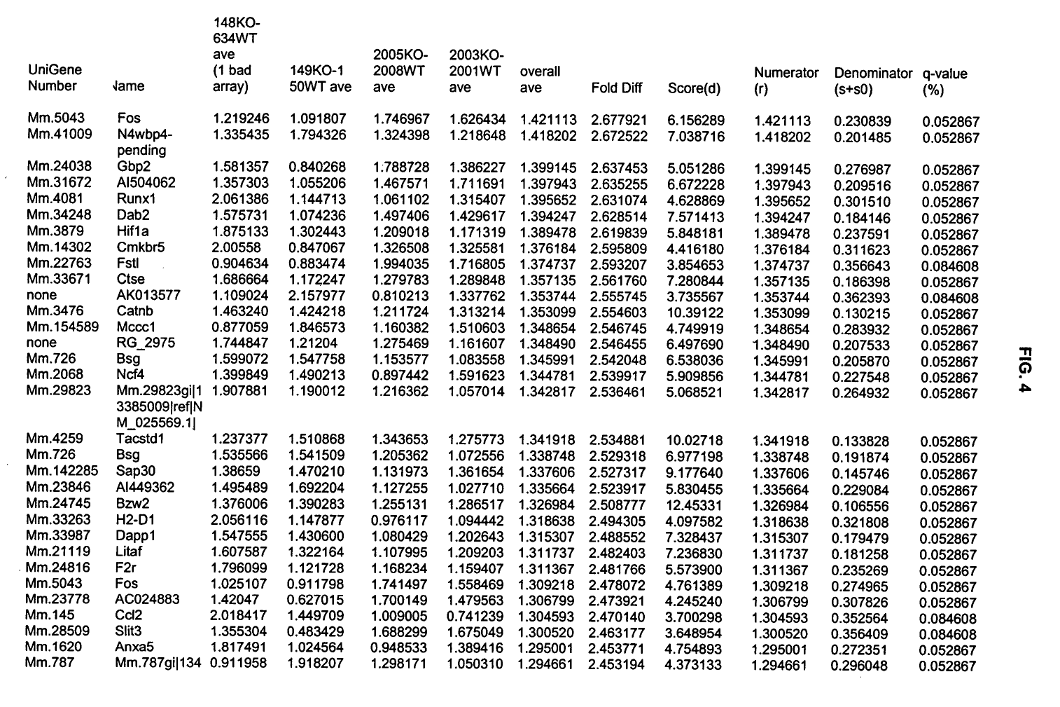

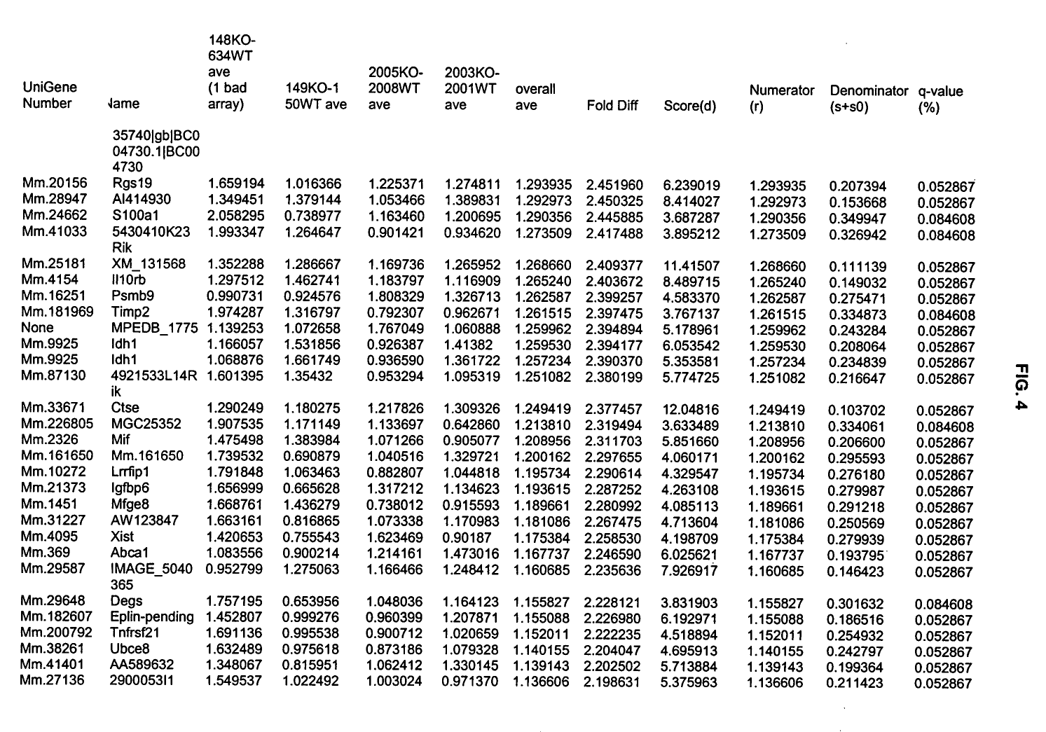

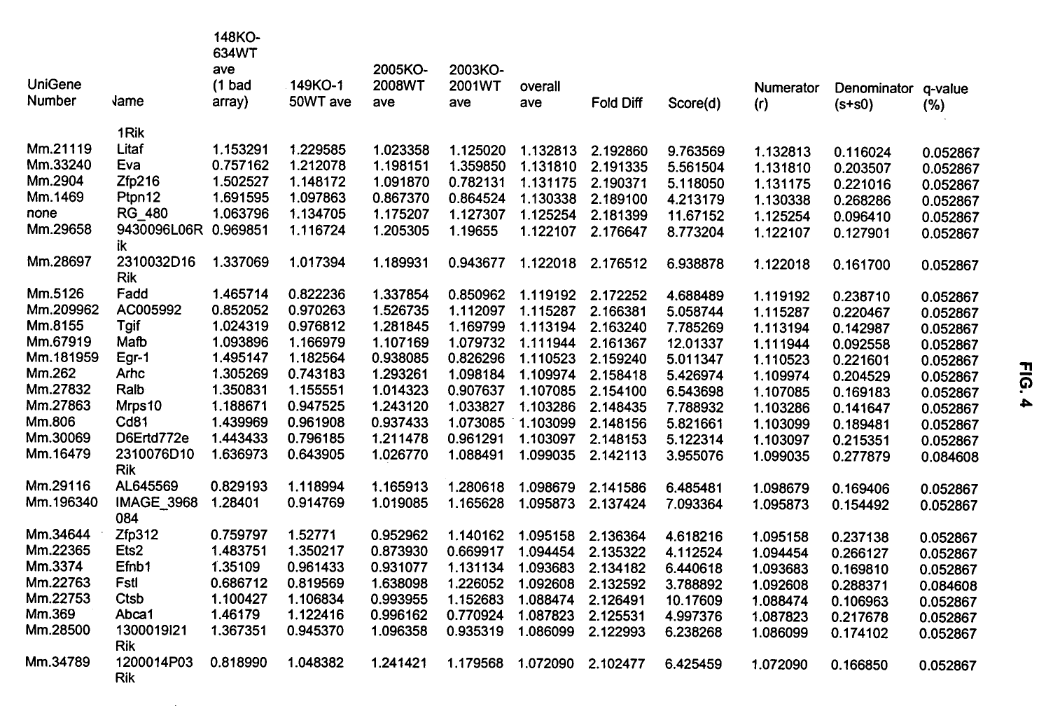

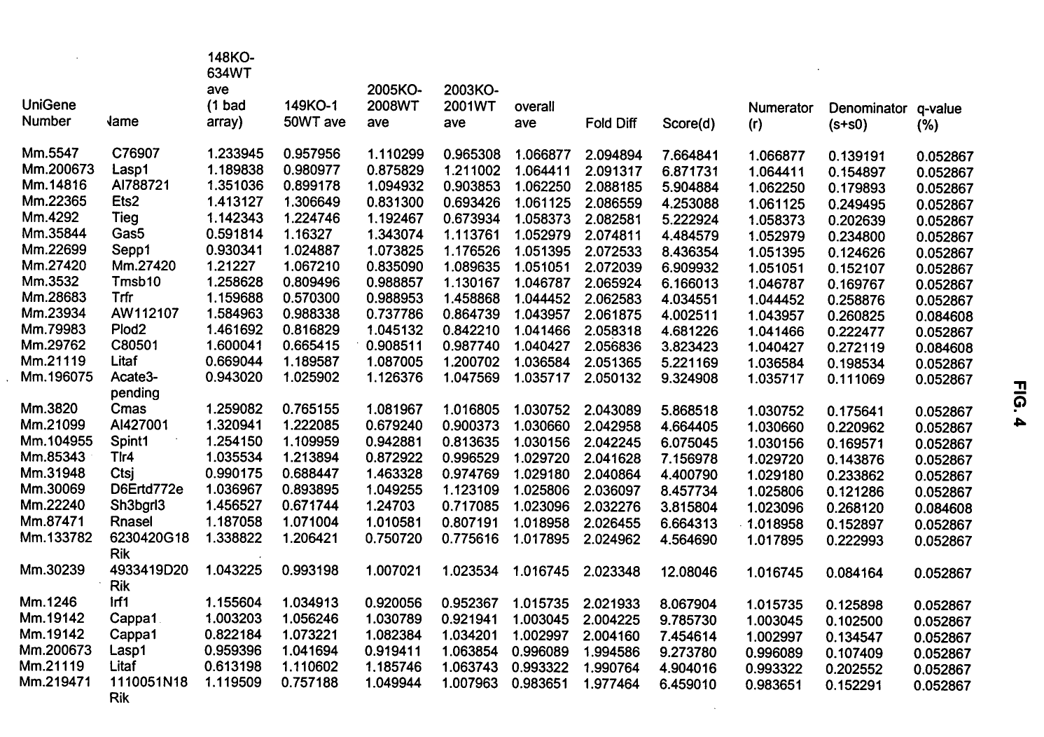

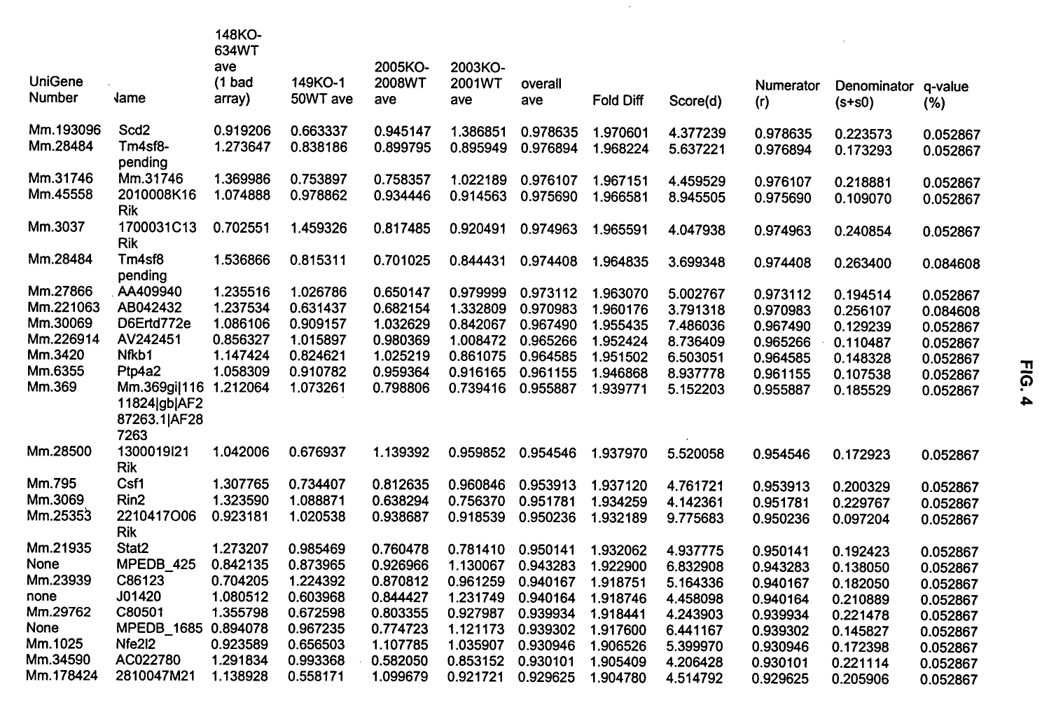

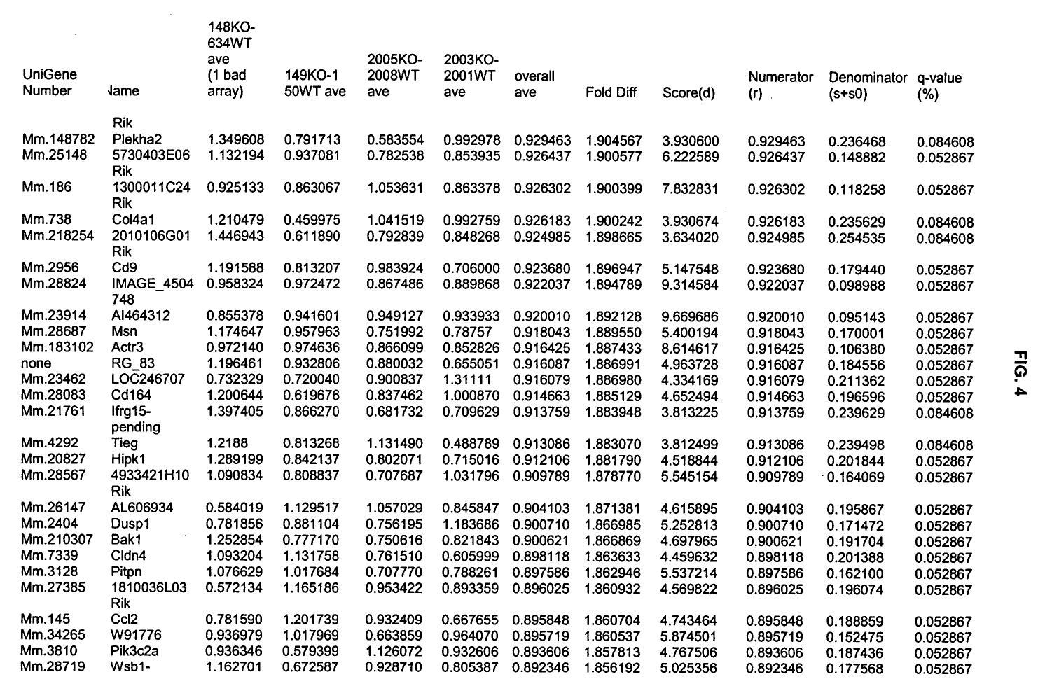

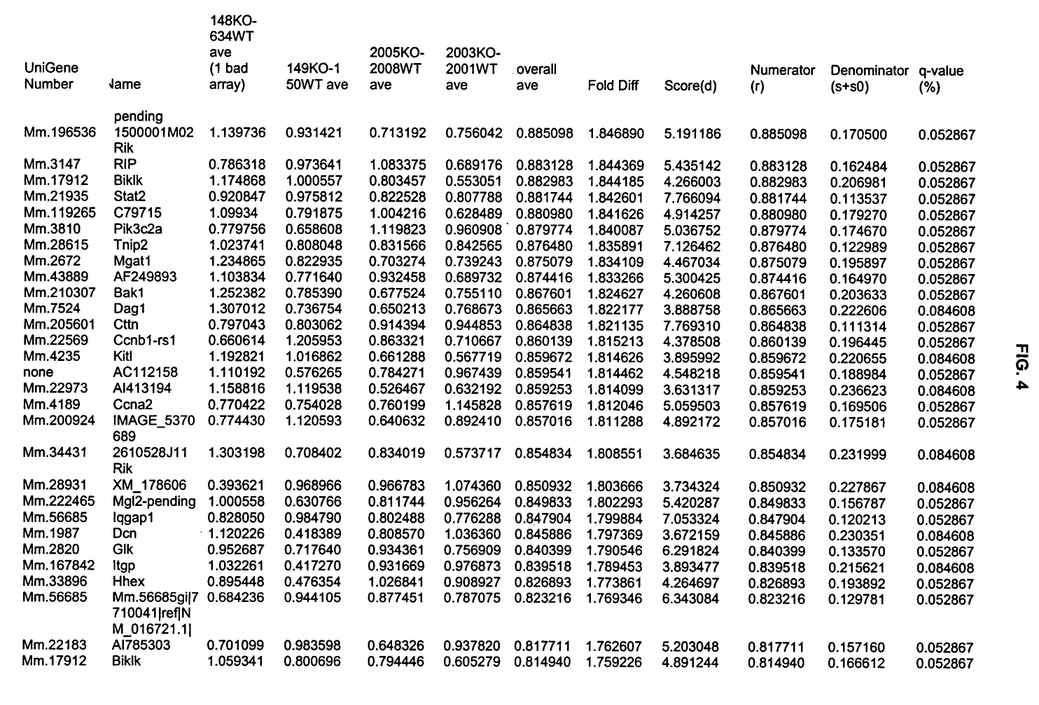

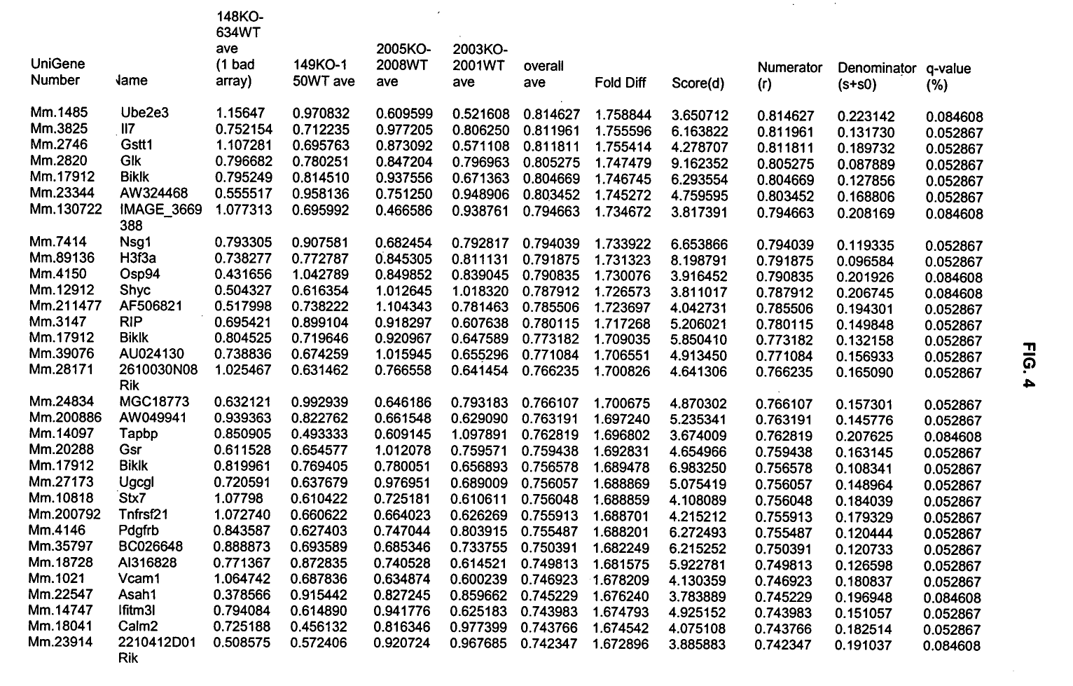

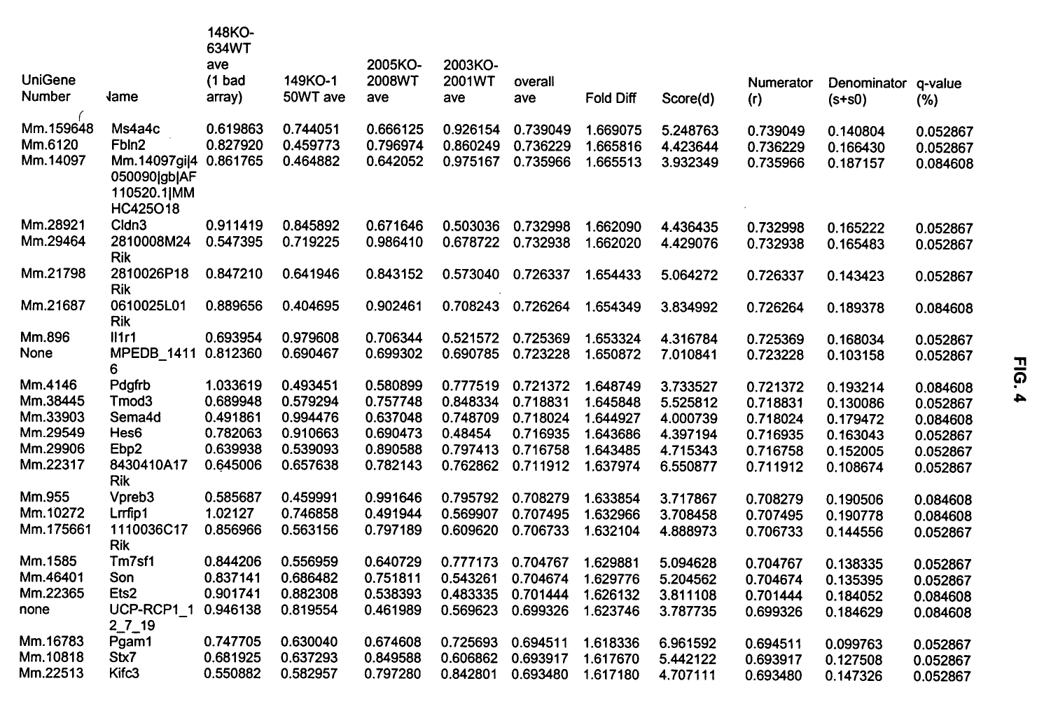

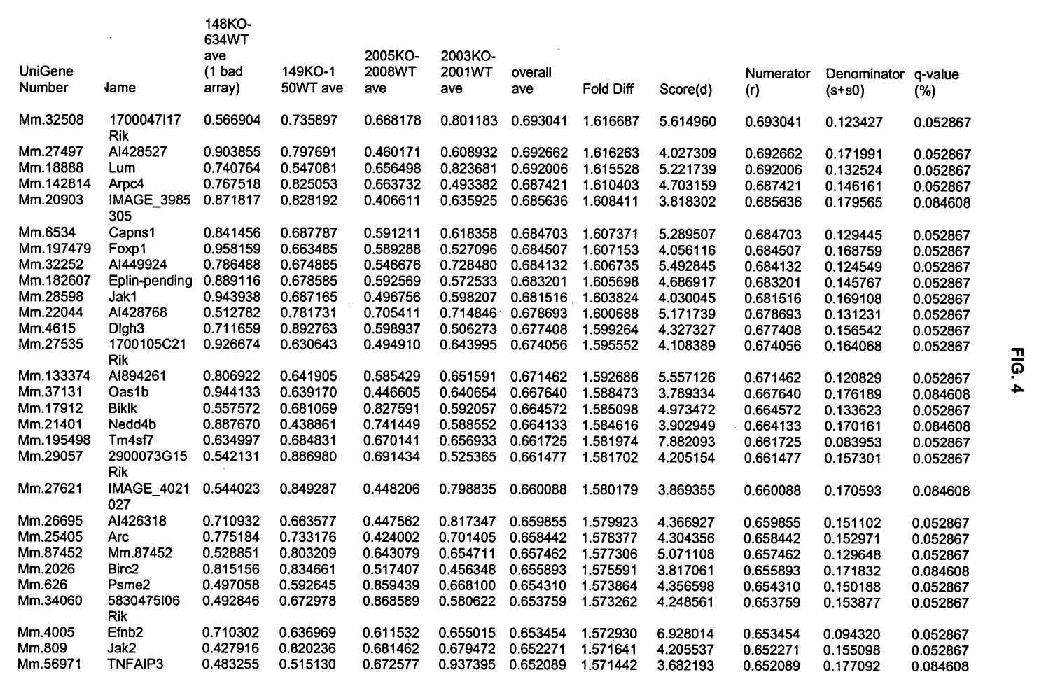

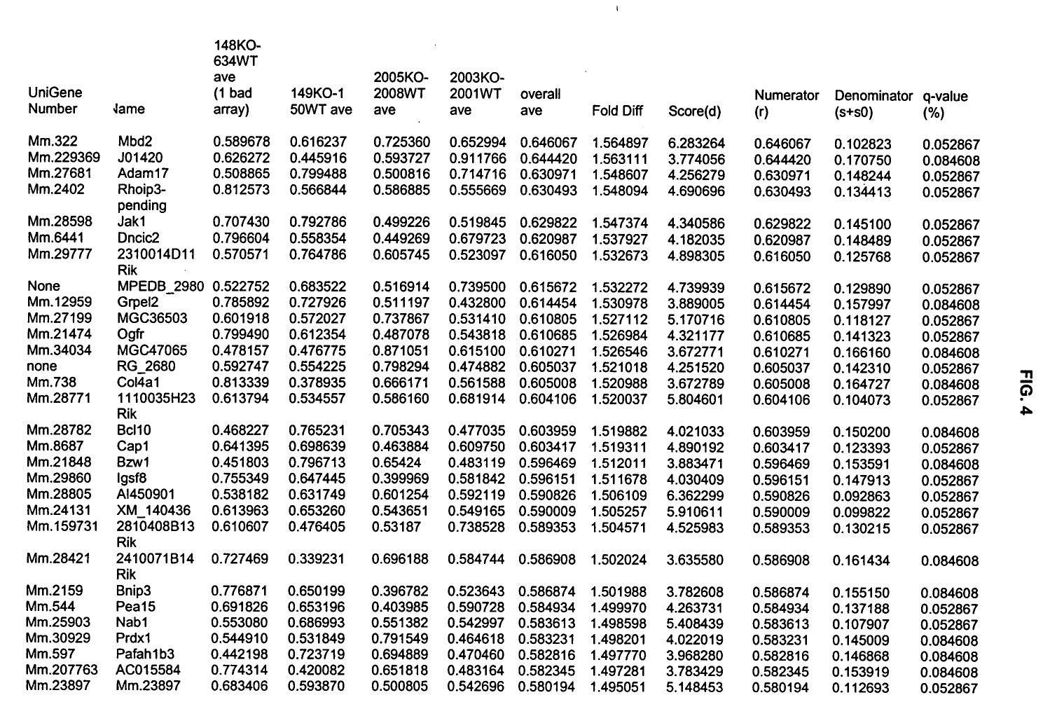

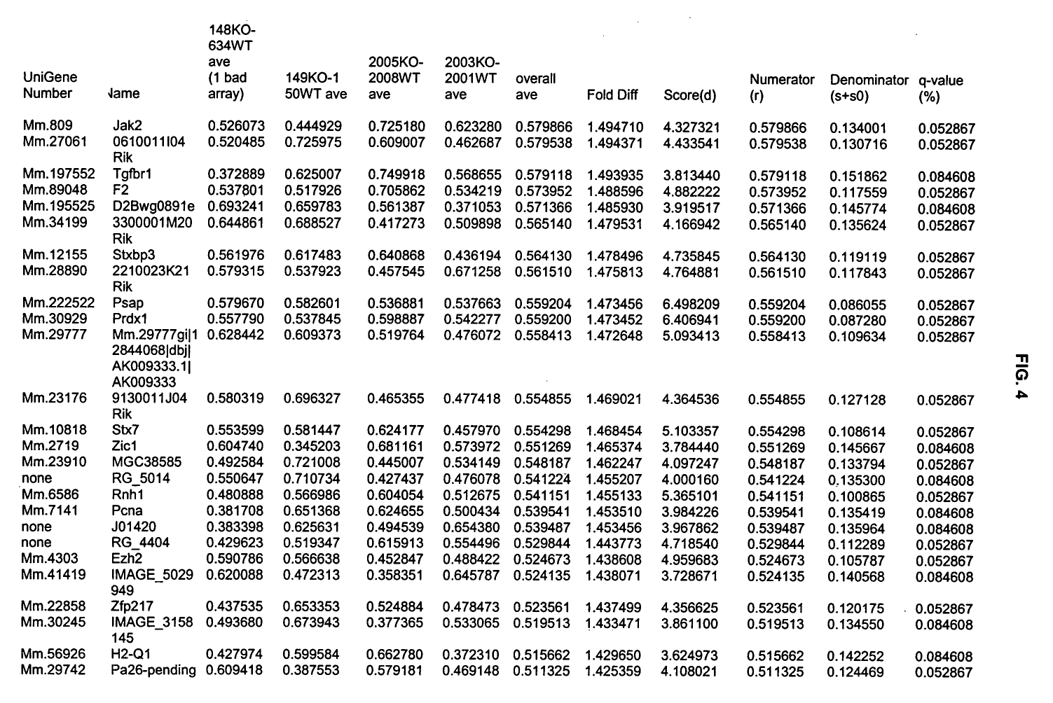

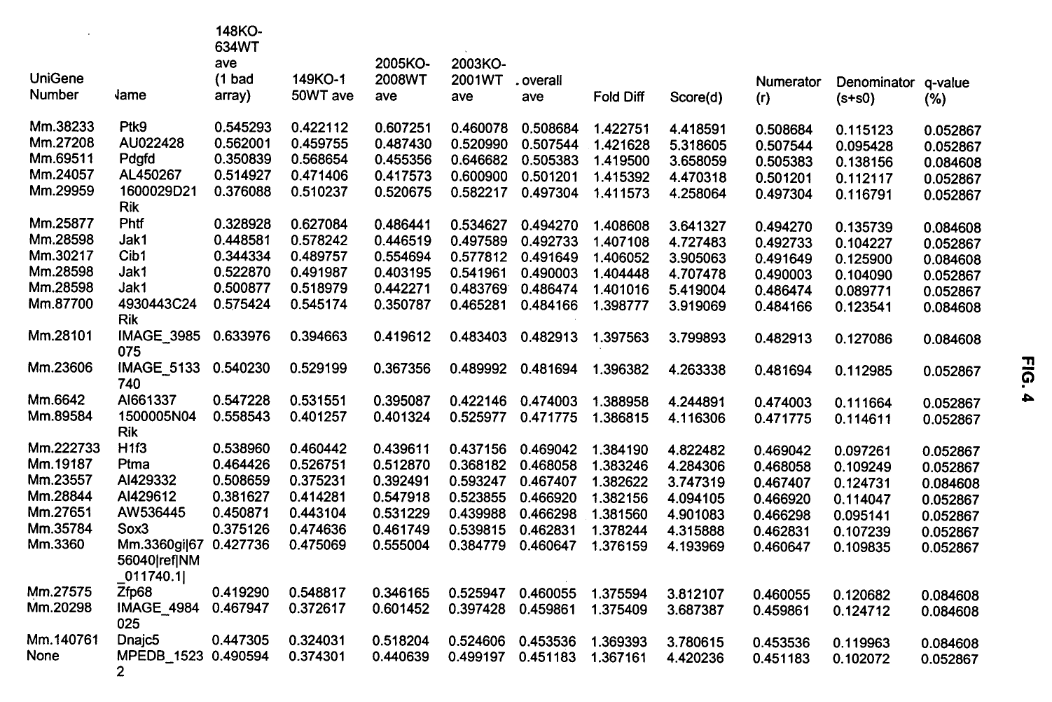

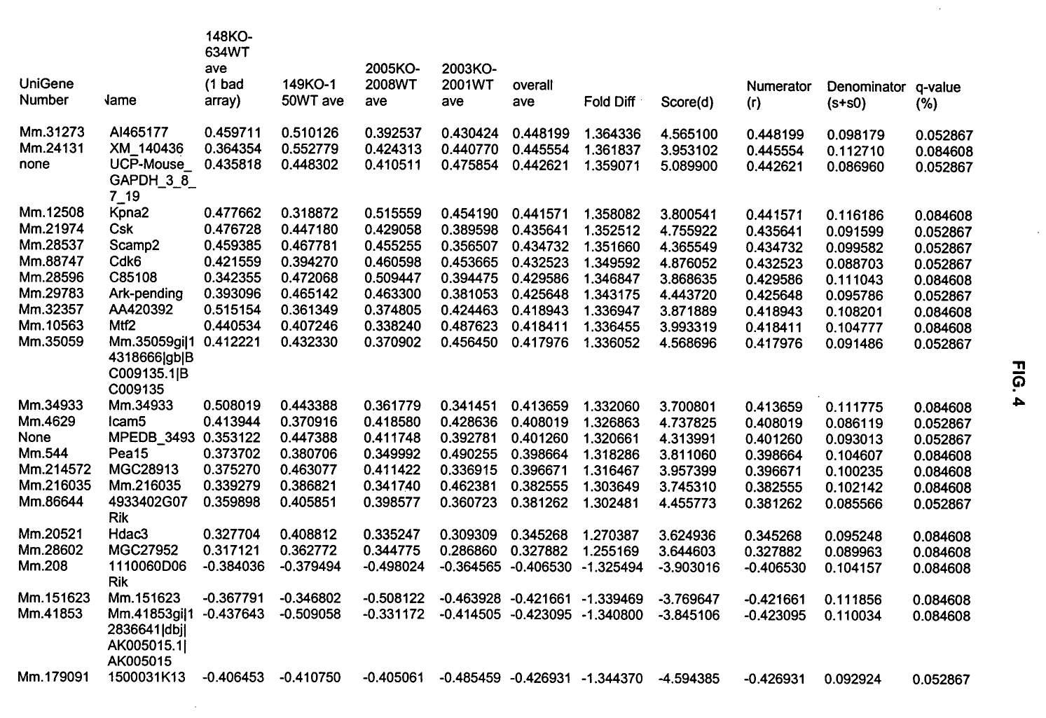

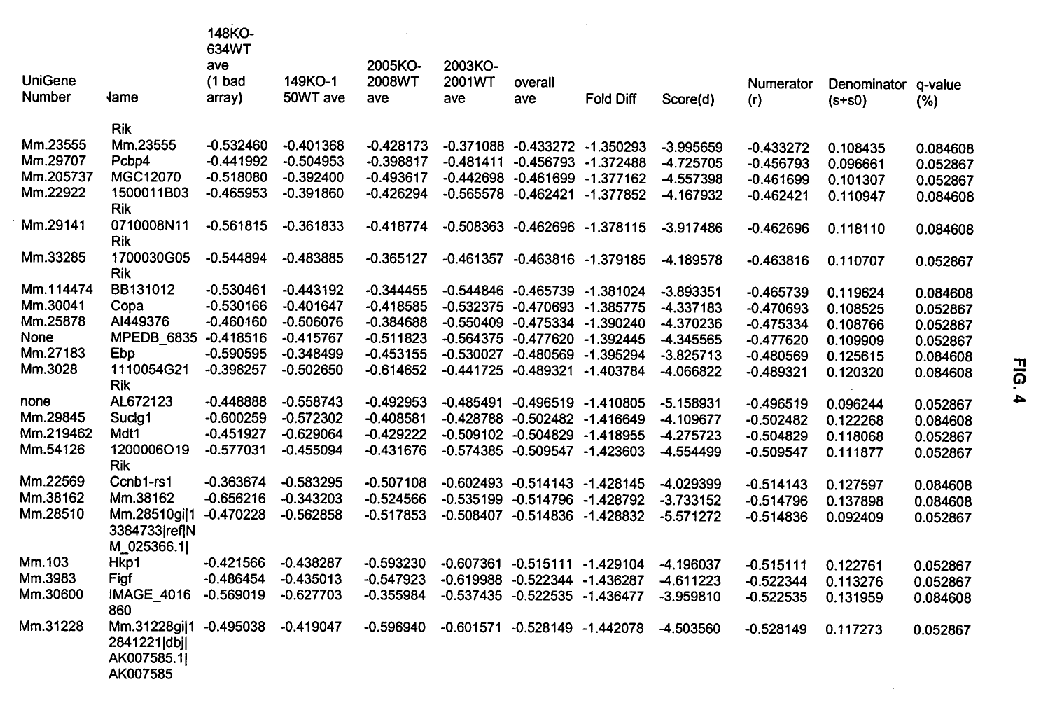

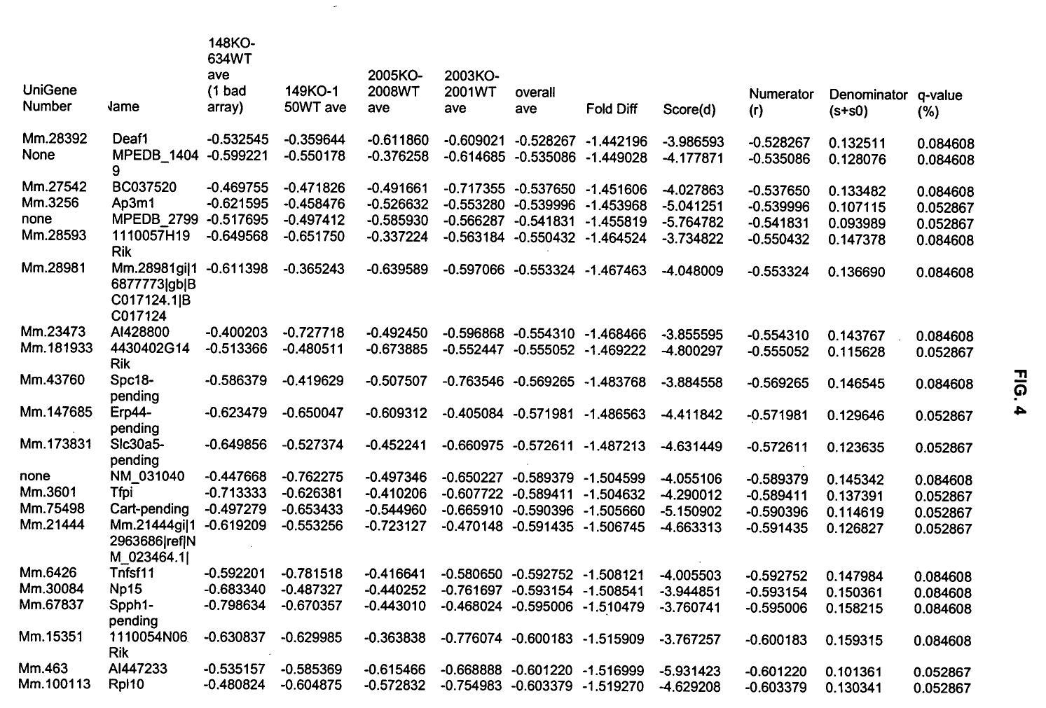

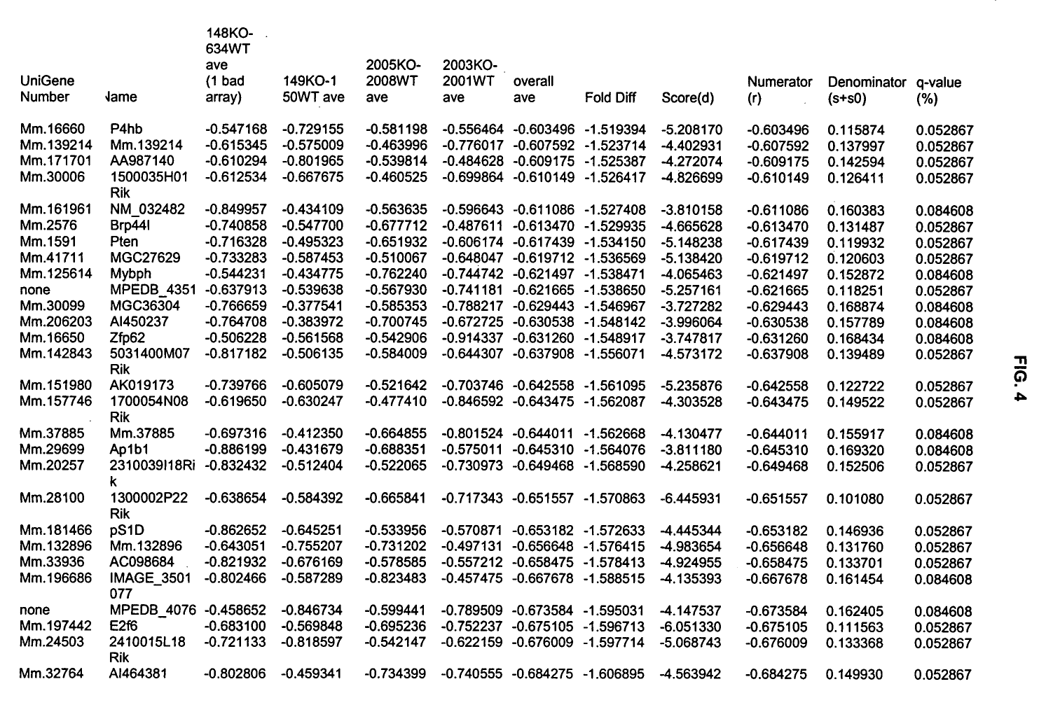

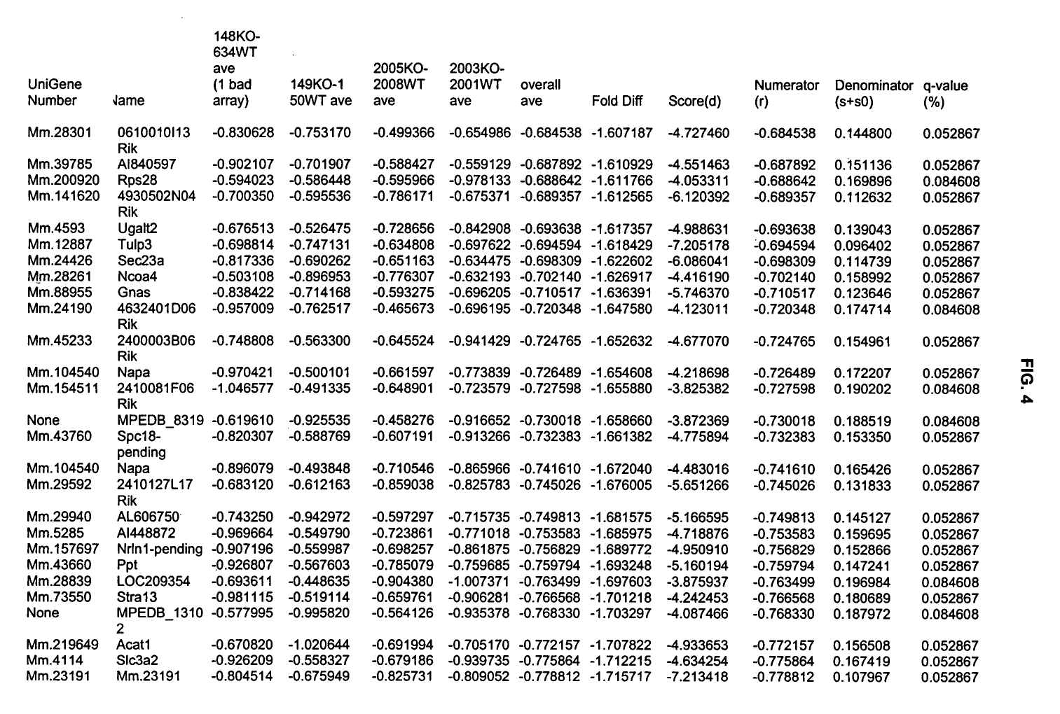

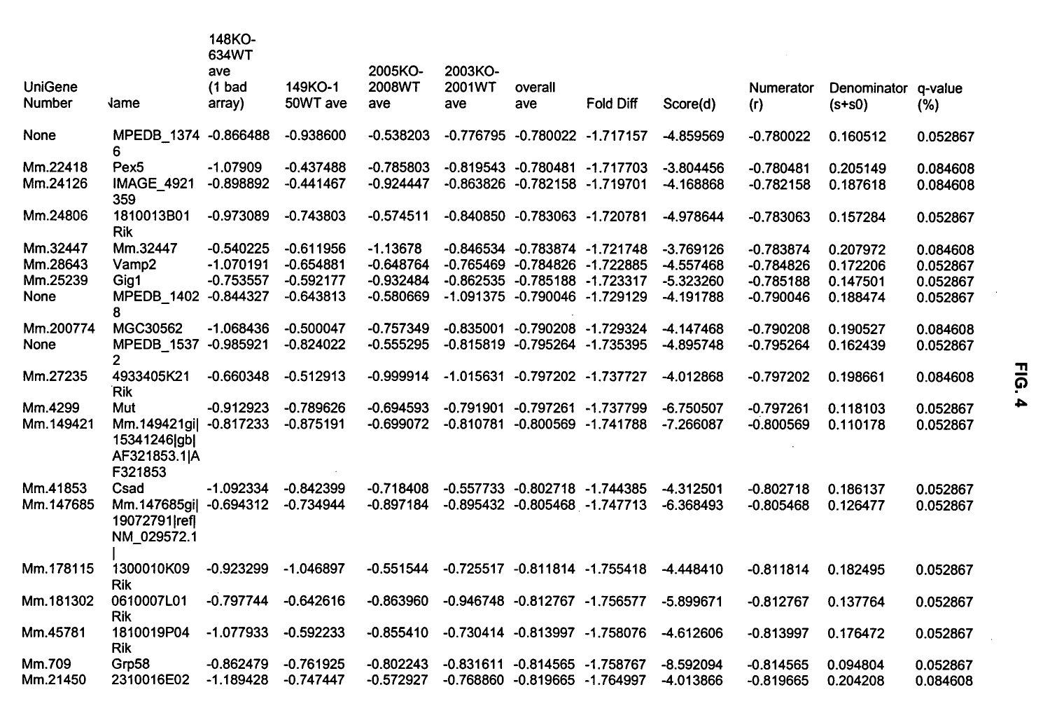

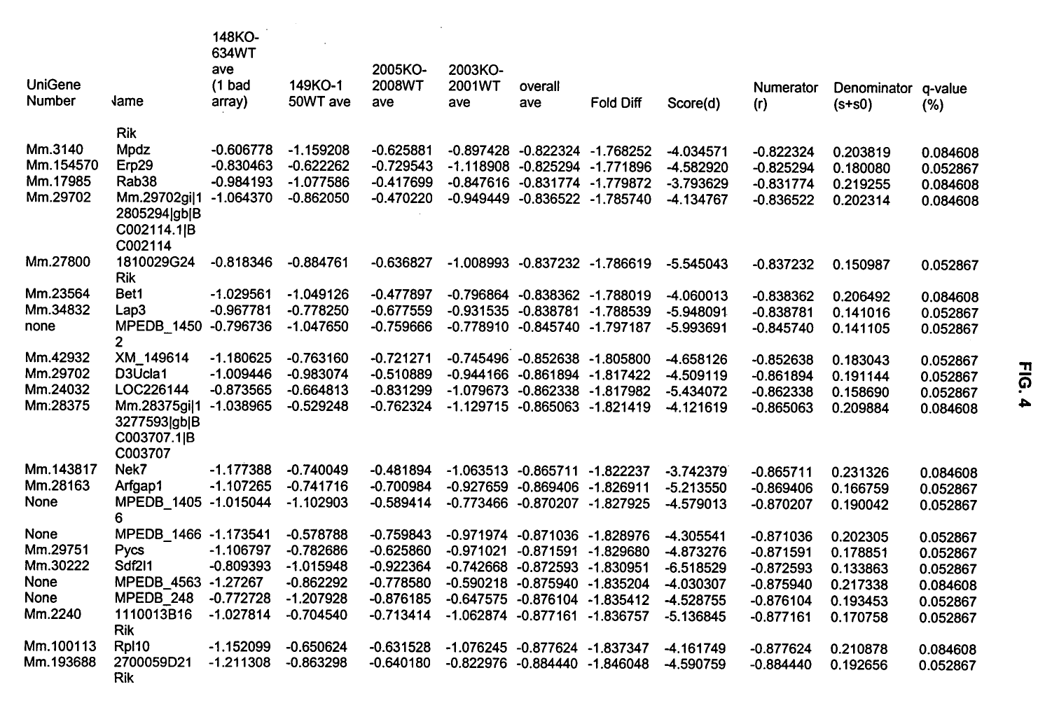

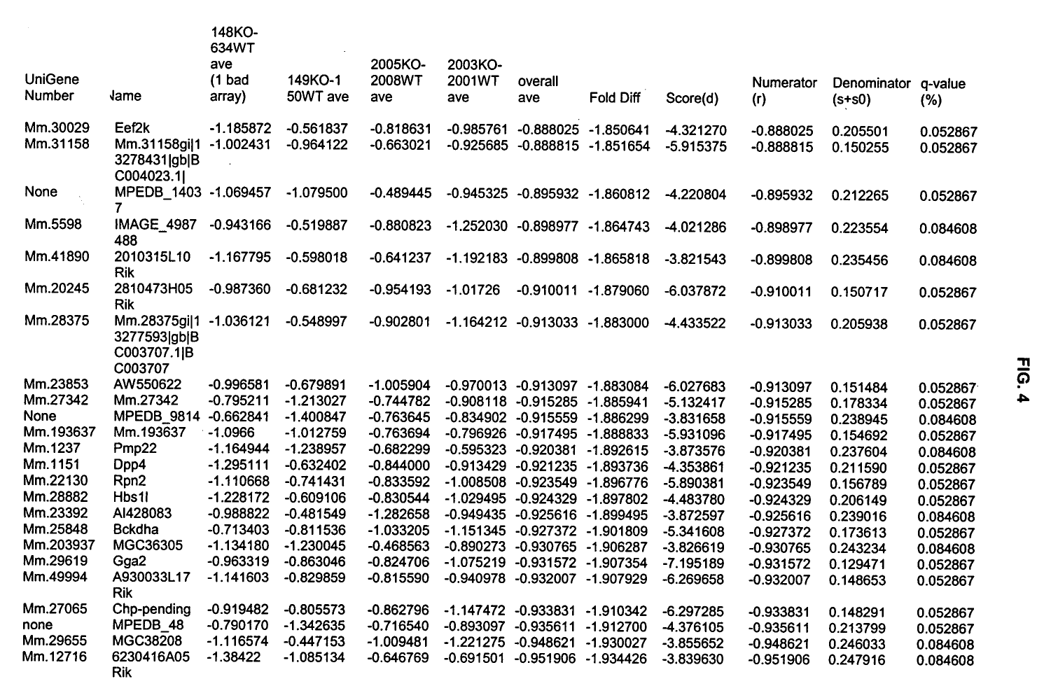

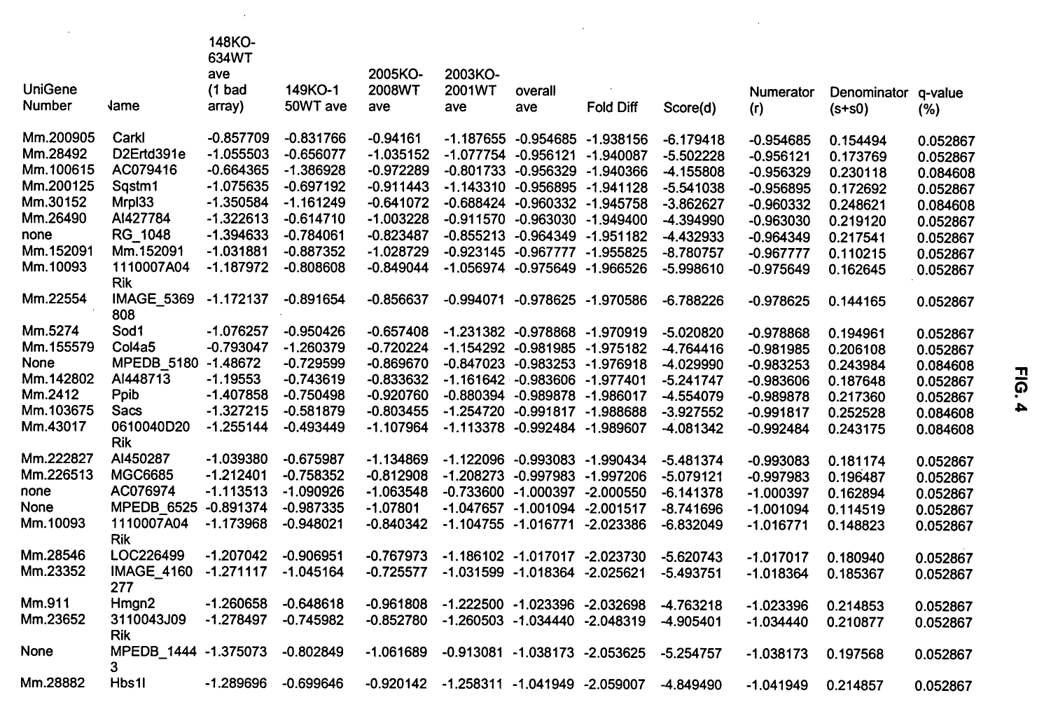

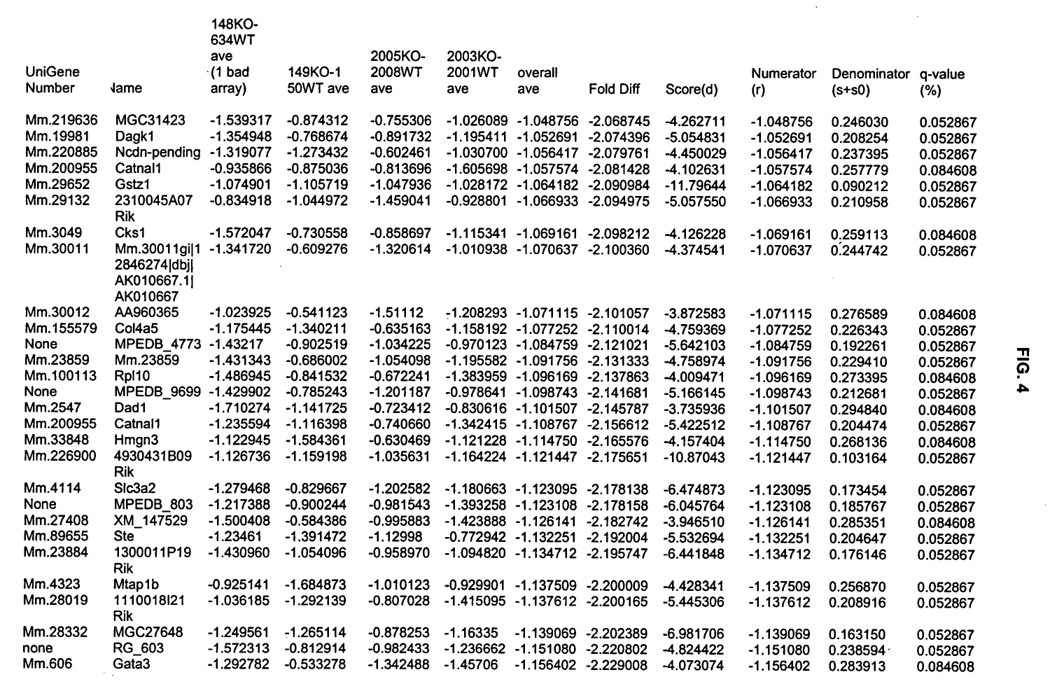

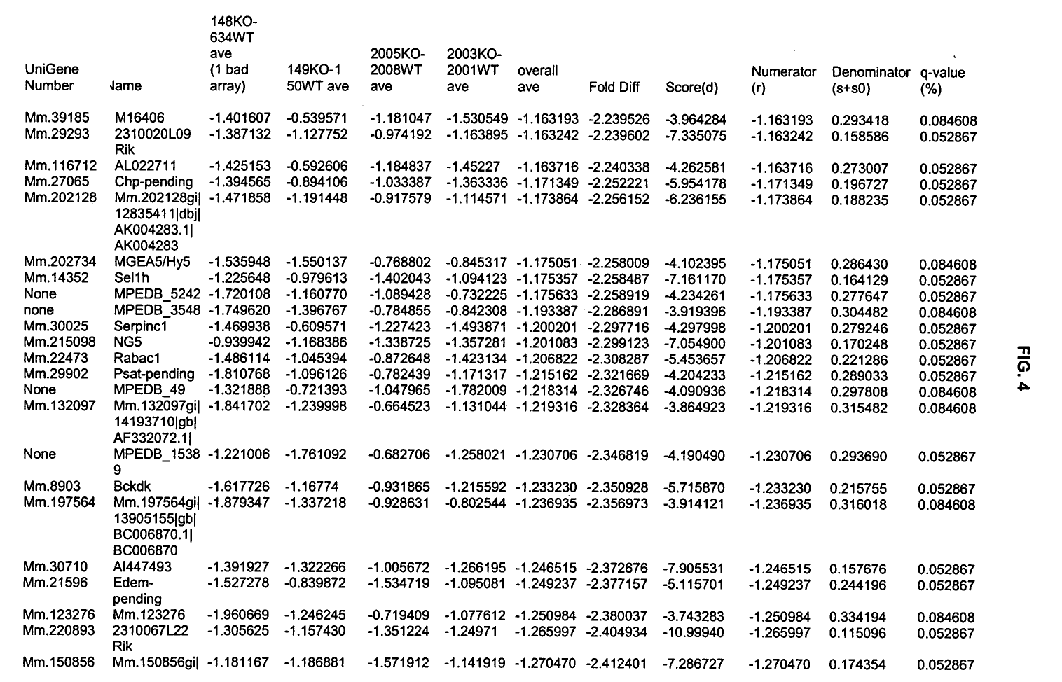

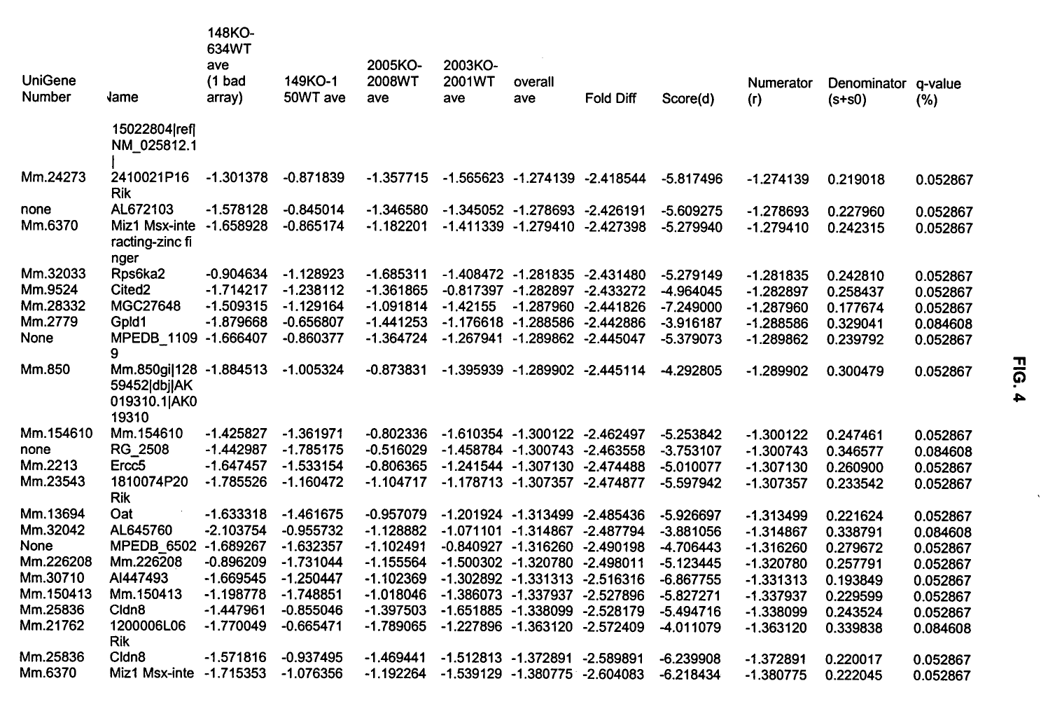

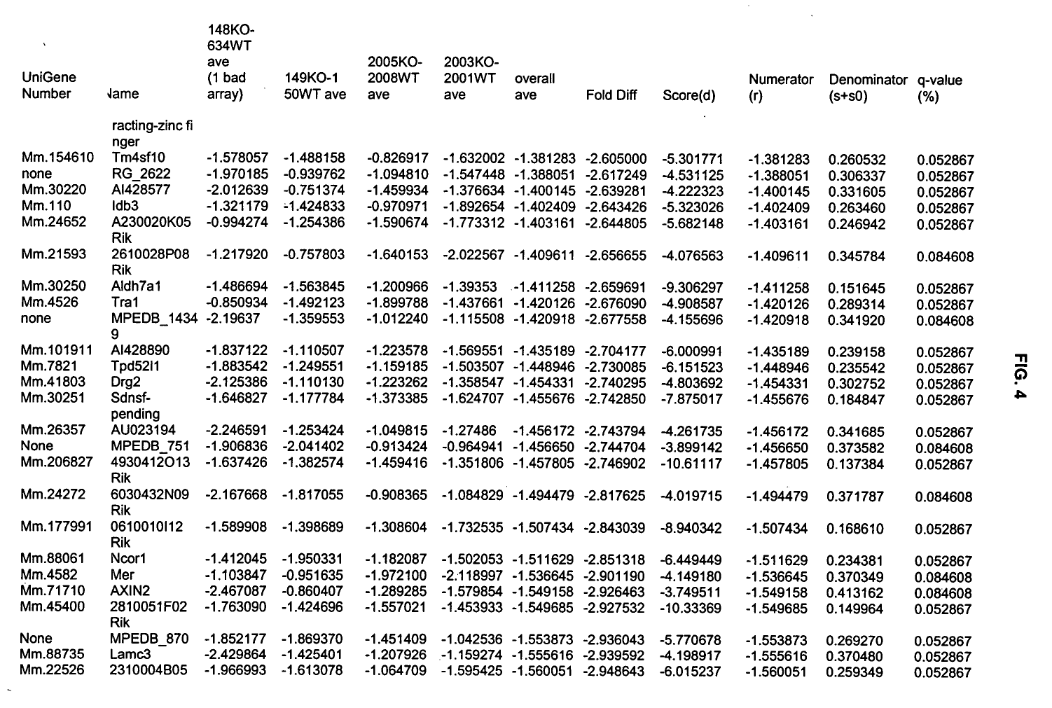

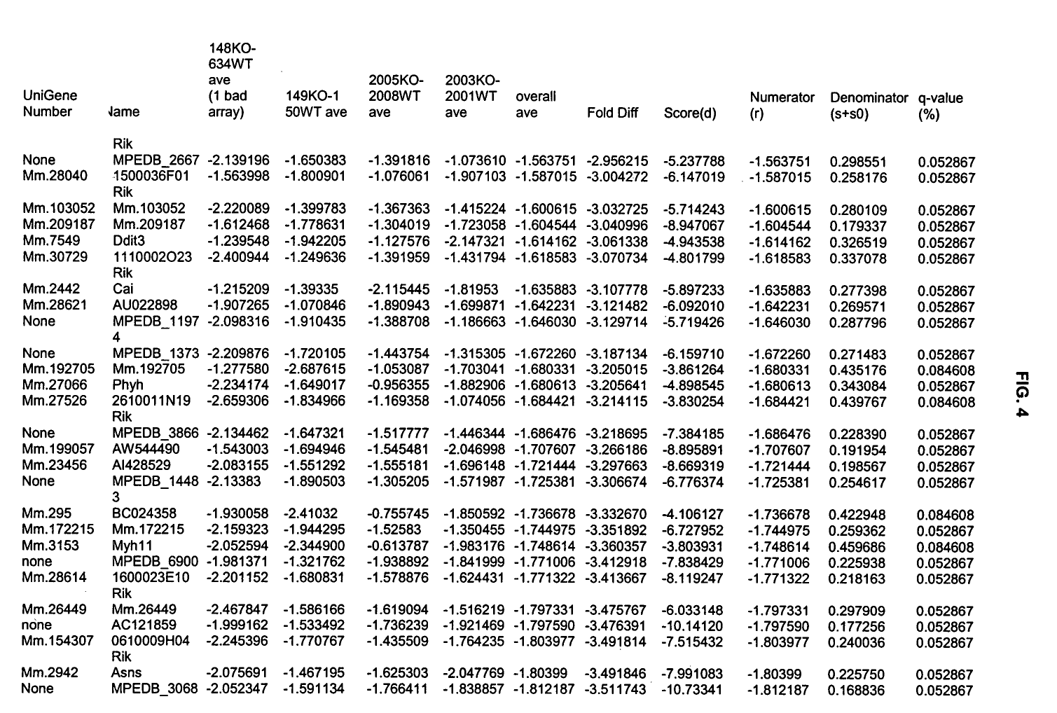

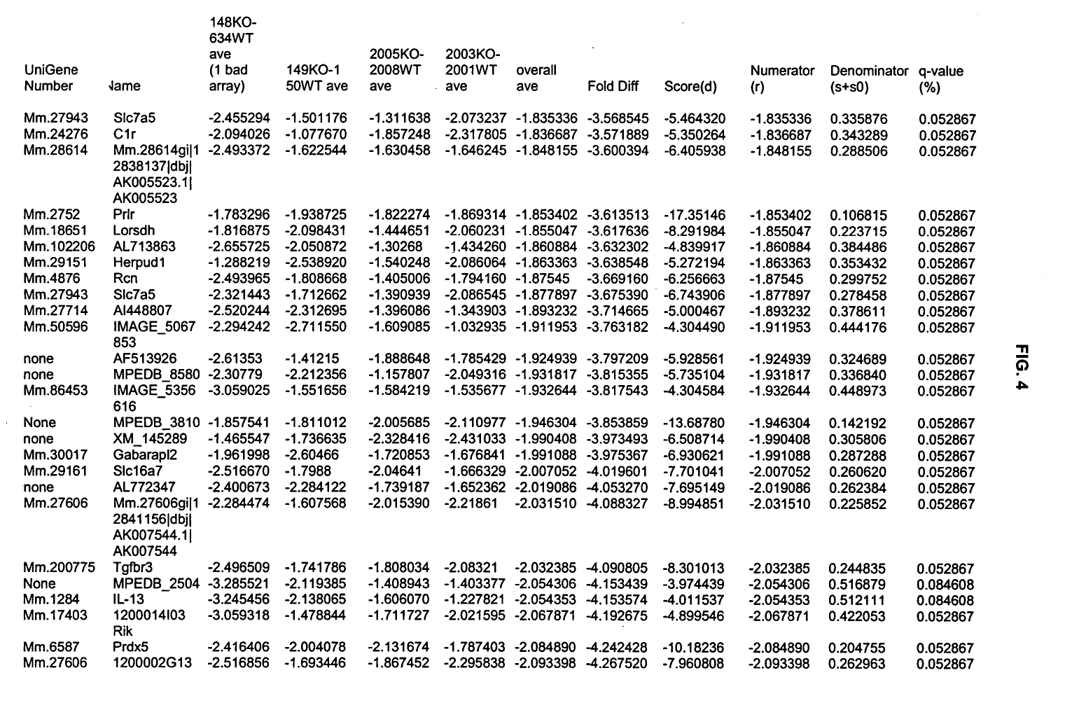

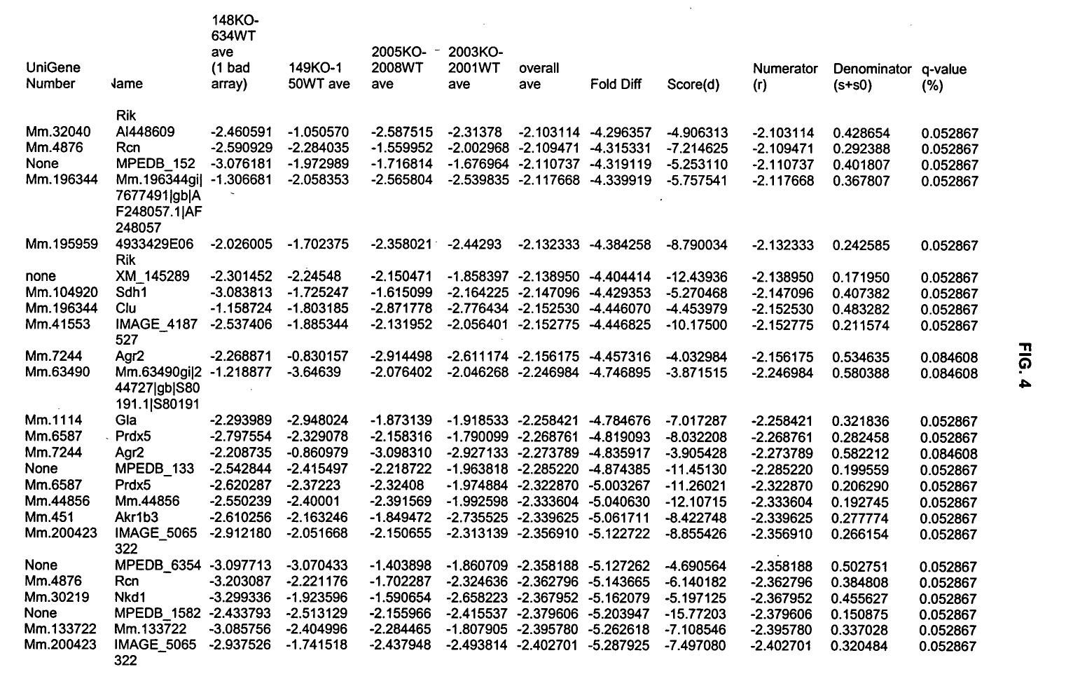

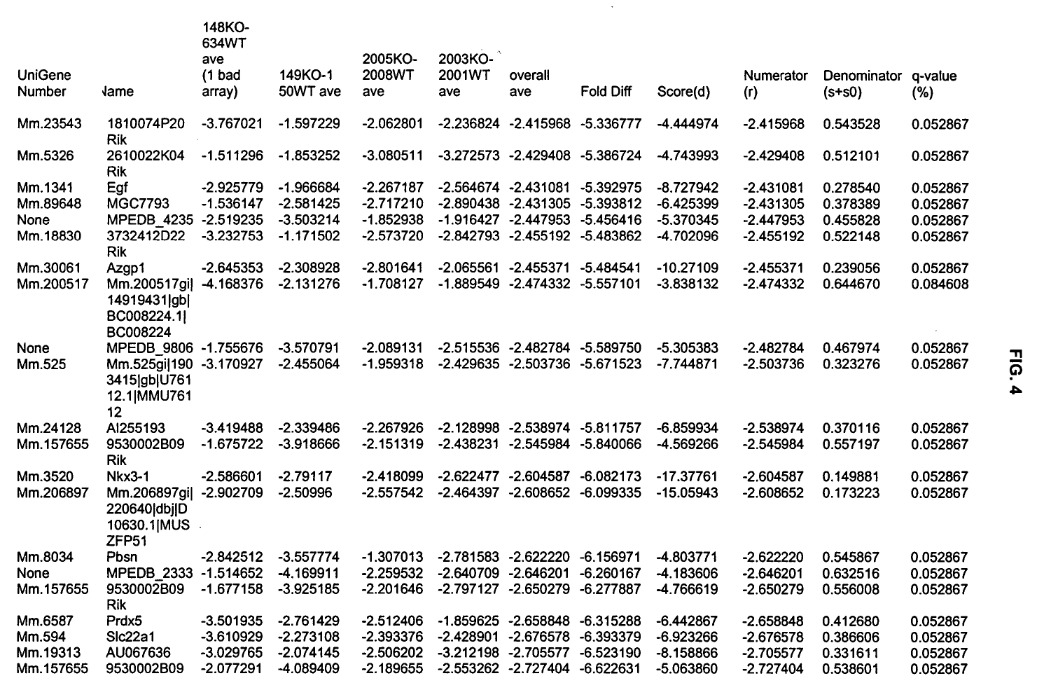

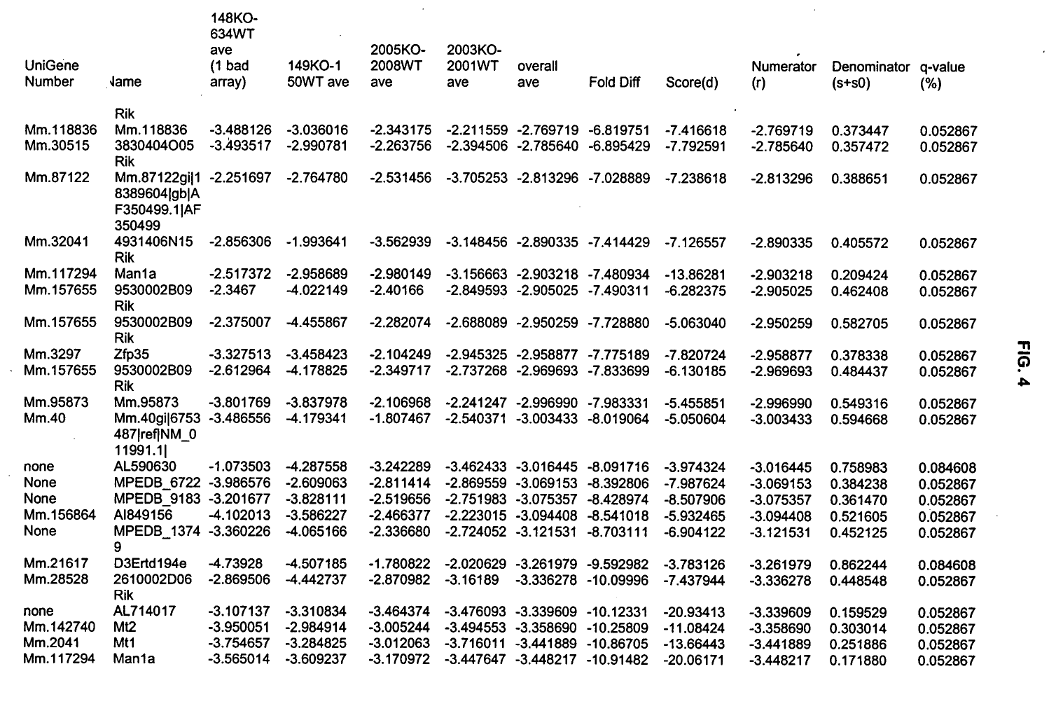

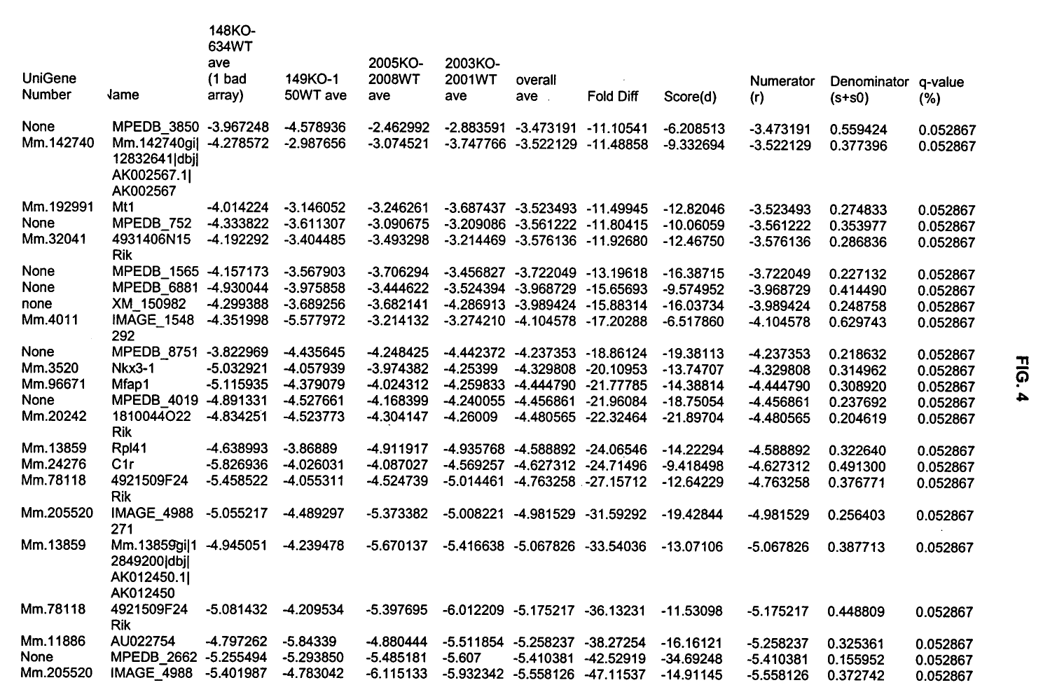

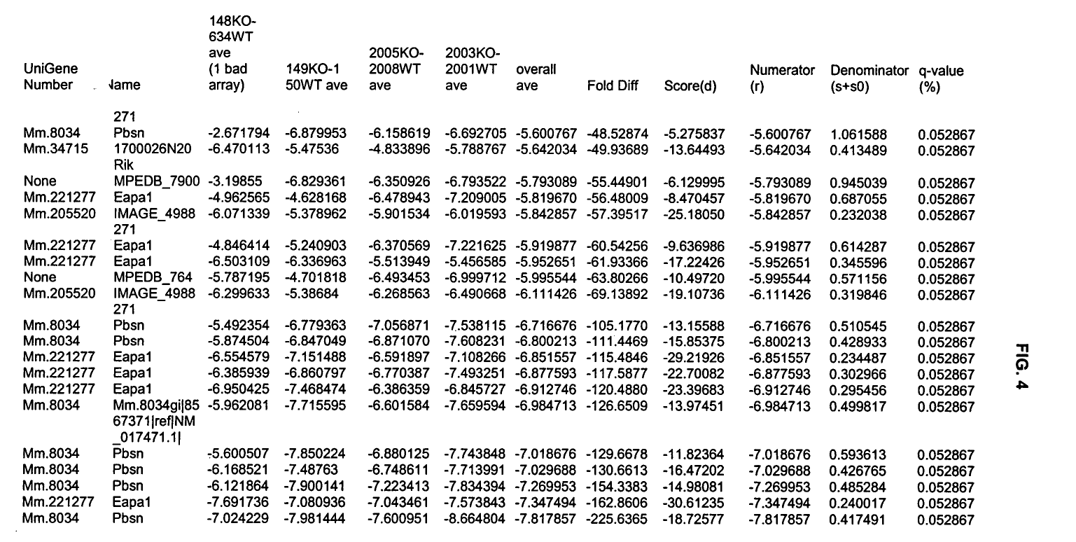

Pten-related prostate cancer biomarkers can be used e.g., for diagnosis or prognostic markers of prostate cancer or as markers of a stage of Pten-related prostate cancer, e.g., PIN, invasive adenocarcinoma, or metastatic carcinoma. Exemplary Pten-related prostate cancer biomarkers are provided in FIGS. 3 and 4. Although the biomarkers in FIGS. 3 and 4 are mouse nucleic acids and proteins, it is expected that human homologues of the murine biomarkers can be used as biomarkers of Pten-related prostate cancer in humans. Thus, the Pten prostate cancer related biomarkers identified in mouse can be used to identify human Pten prostate cancer related biomarkers, that in turn, can be used to diagnose or monitor progression of human prostate cancer, preferably a human prostate cancer that lacks functional PTEN protein in cancer cells.

To identify a biomarker of prostate cancer, an expression pattern or expression profile from a test animal, tissue, or cell is typically compared to an expression pattern or expression profile from a control animal, tissue, or cell. For example, an expression pattern or expression profile from an animal with a prostate specific Pten disruption can be compared to an expression pattern or expression profile from an animal that does not have a prostate specific Pten disruption. In other embodiments, transgenic animals can be used as control. For example, using an animal with androgen independent prostate cancer, an expression pattern or expression profile from an animal, tissue, or cell is compared to an expression pattern or expression profile from a tissue, or cell of a transgenic animal with androgen independent prostate cancer. Expression profiles can also be analyzed before and after androgen ablation or before and after a specific treatment or compound is administered.

In a preferred embodiment, at least one nucleic acid or protein from FIG. 3 is used as a biomarker to diagnose, or monitor prostate cancer. In a further preferred embodiment, a human homolog of at least one nucleic acid or protein from FIG. 3 is used as a biomarker to diagnose, or monitor human prostate cancer. In another preferred embodiment, at least one nucleic acid or protein from FIG. 4 is used as a biomarker to diagnose, or monitor prostate cancer. In a further preferred embodiment, a human homolog of at least one nucleic acid or protein from FIG. 4 is used as a biomarker to diagnose, or monitor human prostate cancer.

Expression Patterns or Expression Profiles

In certain embodiments, Pten-related prostate cancer sequences are identified using expression patterns or expression profiles. An expression pattern of a particular sample is essentially a “fingerprint” of the state of the sample. Typically, an expression pattern is obtained by measuring the products of two or more genes. The evaluation of a number of gene products simultaneously allows the generation of an expression patterns that is characteristic of Pten-related prostate cancer. By comparing expression profiles of Pten-related prostate cancer cells, e.g., directly from animals or from cell culture, to control or normal animals, information regarding which genes are important (including both up- and down-regulation of genes) in Pten-related prostate cancer is obtained.

Expression profiles can be generated for that population of product using any tissue or organ that is associated with Pten-related prostate cancer. For example, expression profiles can be generated from Pten-related metastatic carcinoma cells from any part of the body, e.g., lungs, lymph node or bone. Expression profiles can be generated from e.g., androgen dependent or androgen independent Pten-related prostate cancer.

“Differential expression,” or grammatical equivalents as used herein, refers to qualitative or quantitative differences in the temporal and/or cellular expression patterns within and among cells and tissue. Thus, a differentially expressed gene can qualitatively have its expression and/or activity altered, including an activation or inactivation, in, e.g., tissue from normal-fed versus caloric-restricted animals. Some genes will be expressed in one state or cell type, but not in both. Alternatively, the difference in expression may be quantitative, e.g., in that expression is increased or decreased; i.e., gene expression is either upregulated, resulting in an increased amount of transcript or protein or protein activity, or downregulated, resulting in a decreased amount of transcript or protein or protein activity. The degree to which expression differs need only be large enough to quantify via standard characterization techniques as outlined below, such as by use of Affymetrix GeneChip™ expression arrays (e.g., Lockhart, Nature Biotechnology 14:1675-1680, 1996). Other techniques for anlaysing levels of nucleic acids include, but are not limited to, quantitative reverse transcriptase PCR, northern analysis and RNase protection.

The effects of compounds that affect a Pten-related prostate cancer or Pten-related disregulated cell growth can be assessed using a variety of assays. Such assays include at least one of the changes in RNA levels, changes in protein levels, changes in protein activity levels, changes in carbohydrate or lipid levels, changes in nucleic acid levels, changes in rate of protein or nucleic acid synthesis, changes in protein or nucleic acid stability, changes in protein or nucleic acid accumulation levels, changes in protein or nucleic acid degradation rate, and changes in protein or nucleic acid structures or function.

Assays for performing such analyses are well known in the art. For example, assay for the activity of a protein activity, e.g., a phosphatase, a transcription factor, a kinase, an enzyme involved in glucose metabolism can be performed using a known assay, such as measuring the ability to modulate transcription, modulate phosphorylation, or perform an enzymatic reaction.

Control data can be obtained from a prior study, the results of which are recorded, as opposed to obtaining the control data concurrently, e.g, at the same time a test intervention is being evaluated. Thus, the control data may be obtained from an administering of a diet program which was previously performed in a normal or dwarf subject. This control data may be obtained once and stored for recall in later screening studies for comparison against the results in the later screening studies.

Identification via Homology or Linkage

Additional Pten-related prostate cancer sequences can be identified by substantial nucleic acid and/or amino acid sequence homology or linkage to the Pten-related prostate cancer sequences outlined herein. Such homology can be based upon the overall nucleic acid or amino acid sequence, and is generally determined as outlined below, using either homology programs or hybridization conditions.

The Pten-related prostate cancer nucleic acid and protein sequences of the invention, e.g., the sequences in FIG. 3, can be fragments of larger genes, i.e., they are nucleic acid segments. “Genes” in this context includes coding regions, non-coding regions, and mixtures of coding and non-coding regions. Accordingly, as will be appreciated by those in the art, using the sequences provided herein, extended sequences, in either direction, of the Pten-related prostate cancer genes can be obtained, using techniques well known in the art for cloning either longer sequences or the full length sequences; see Ausubel, et al., supra. Much can be done by informatics and many sequences can be clustered to include multiple sequences corresponding to a single gene, e.g., systems such as UniGene (see, www.ncbi.nlm.nih.gov/unigene/).

Screening Assay for Expression Pattern—High Throughput Screening

In some embodiments, the expression pattern of multiple Pten-related prostate cancer genes in animals that comprise a prostate specific Pten homozygous disruption, or in biological samples exposed to a potential intervention, are assayed using high-throughput technology.

Often, the expression pattern is obtained by monitoring levels of RNA expression, e.g., levels of mRNA. RNA expression monitoring can be performed on a single polynucleotide or simultaneously for a number of polynucleotides. For example, an oligonucletide array may be used. Other methods, e.g., PCR techniques for measurement of gene expression levels can also be used. Often, once a candidate drug or intervention is identified using high throughput analysis, the results is further confirmed using an alternative method of analyzing expression pattern changes. For example, if an oligonucleotide array is used to initially screen a test intervention, those that identify a test compound or intervention that induces an expression pattern that mimics that observed in caloric restriction, dwarfism, or both, another assay such as a PCR assay can be performed to confirm the results.

Nucleic Acid Probes

In one embodiment, nucleic acid probes to biomarker nucleic acid are made. The nucleic acid probes are designed to be substantially complementary to the biomarker nucleic acids, i.e. the target sequence (either the target sequence of the sample or to other probe sequences, e.g., in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under appropriate reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, from about 10 to about 80 bases, or from about 30 to about 50 bases. That is, generally complements of ORFs or whole genes are not used. In some embodiments, nucleic acids of lengths up to hundreds of bases can be used.

In some embodiments, more than one probe per sequence is used, with either overlapping probes or probes to different sections of the target being used. That is, two, three, four or more probes, with three being preferred, are used to build in a redundancy for a particular target. The probes can be overlapping (i.e., have some sequence in common), or separate. In some cases, PCR primers may be used to amplify signal for higher sensitivity.

Attachment of the Target Nucleic Acids to the Solid Support

In some embodiments, as noted above, arrays are used in the screening assays. The arrays can, e.g., be generated to comprise probes for multiple biomarkers associated with Pten-related prostate cancer.

In general, the probes are attached to a biochip in a wide variety of ways, as will be appreciated by those in the art. As described herein, the nucleic acids can either be synthesized first, with subsequent attachment to the biochip, or can be directly synthesized on the biochip.

In this embodiment, oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

Biochips

The biochip comprises a suitable solid substrate. By “substrate” or “solid support” or other grammatical equivalents herein is meant a material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, etc. In general, the substrates allow optical detection and do not appreciably fluoresce. One such substrate is described in copending application entitled Reusable Low Fluorescent Plastic Biochip, U.S. application Ser. No. 09/270,214, filed Mar. 15, 1999, herein incorporated by reference in its entirety.

Generally the substrate is planar, although as will be appreciated by those in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In one embodiment, the surface of the biochip and the probe may be derivatized with chemical functional groups for subsequent attachment of the two. Thus, e.g., the biochip is derivatized with a chemical functional group including, but not limited to, amino groups, carboxy groups, oxo groups and thiol groups. Using these functional groups, the probes can be attached using functional groups on the probes. For example, nucleic acids containing amino groups can be attached to surfaces comprising amino groups, e.g., using linkers as are known in the art; e.g., homo-or hetero-bifunctional linkers as are well known (see, 1994 Pierce Chemical Company catalog, technical section on cross-linkers, pages 155-200). In addition, in some cases, additional linkers, such as alkyl groups (including substituted and heteroalkyl groups) may be used.

Hybridization and Sandwich Assays

Nucleic acid assays can be detected hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, attached to a solid support, and then the labeled probe is added under conditions that allow the formation of a hybridization complex.

A variety of hybridization conditions may be used in the present invention, including high, moderate and low stringency conditions as outlined above. The assays are generally run under stringency conditions which allow formation of the label probe hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways. Components of the reaction may be added simultaneously, or sequentially, in different orders, with certain embodiments outlined below. In addition, the reaction may include a variety of other reagents. These include salts, buffers, neutral proteins, e.g., albumin, detergents, etc. which may be used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

Detection of Labeled Target Nucleic Acid Bound to Immobilized Probe

One of skill will readily appreciate that methods similar to those in the preceding section can be used in embodiments where the a nucleic acid to be examined is attached to a solid support and labeled probe is used to detect the biomarker nucleic acid.

Amplification-Based Assays

Amplification-based assays can also be used measure the expression level of biomarker sequences. These assays are typically performed in conjunction with reverse transcription. In such assays, a biomarker nucleic acid sequence acts as a template in an amplification reaction (e.g., Polymerase Chain Reaction, or PCR). In a quantitative amplification, the amount of amplification product will be proportional to the amount of template in the original sample. Comparison to appropriate controls provides a measure of the amount of biomarker RNA. Methods of quantitative amplification are well known to those of skill in the art. Detailed protocols for quantitative PCR are provided, e.g., in Innis et al, PCR Protocols, A Guide to Methods and Applications (1990).

In some embodiments, a TaqMan based assay is used to measure expression. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, e.g., literature provided by Perkin-Elmer, e.g., www2.perkin-elmer.com).

Other suitable amplification methods include, but are not limited to, ligase chain reaction (LCR) (see Wu & Wallace, Genomics 4:560 (1989), Landegren et al., Science 241:1077 (1988), and Barringer et al., Gene 89:117 (1990)), transcription amplification (Kwoh et al., Proc. Natl. Acad. Sci. USA 86:1173 (1989)), self-sustained sequence replication (Guatelli et al., Proc. Nat. Acad. Sci. USA 87:1874 (1990)), dot PCR, and linker adapter PCR, etc.

Methods of Assaying Protein Expression Levels

The expression levels of multiple proteins can also be performed. Similarly, these assays may also be performed on an individual basis.

In another method, antibodies to the biomarker protein find use in in situ imaging techniques for detection of the protein(s), e.g., in histology (e.g., Methods in Cell Biology: Antibodies in Cell Biology, volume 37 (Asai, ed. 1993)). In this method cells are contacted with from one to many antibodies to the protein(s). Following washing to remove non-specific antibody binding, the presence of the antibody or antibodies is detected. In one embodiment, the antibody is detected by incubating with a secondary antibody that contains a detectable label, e.g., multicolor fluorescence or confocal imaging. In another method the primary antibody to the protein(s) contains a detectable label, e.g., an enzyme marker that can act on a substrate. In another embodiment each one of multiple primary antibodies contains a distinct and detectable label. This method finds particular use in simultaneous screening for a plurality of proteins. Many other histological imaging techniques are also provided by the invention.

In one embodiment the label is detected in a fluorometer which has the ability to detect and distinguish emissions of different wavelengths. In addition, a fluorescence activated cell sorter (FACS) can be used in the method.

VIII. Pharmaceutical Compositions and Administration

Compounds or treatments that have an effect on a Pten-related prostate cancer or Pten-related disregulated cell growth can be administered directly to the patient for inhibition of cancer, tumor, or precancer cells in vivo. Administration is by any of the routes normally used for introducing a compound into ultimate contact with the tissue to be treated. The compounds are administered in any suitable manner, preferably with pharmaceutically acceptable carriers. Suitable methods of administering such compounds are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17^{th} ed. 1985)). For example, if in vivo delivery of a biologically active Pten-related prostate cancer protein is desired, the methods described in Schwarze et al. (see Science 285:1569-1572 (1999)) can be used.

The compounds (nucleic acids, proteins, and modulators), alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a patient, in the context of the present invention should be sufficient to effect a beneficial therapeutic response in the patient over time. The dose will be determined by the efficacy of the particular compound employed and the condition of the patient, as well as the body weight or surface area of the patient to be treated. The size of the dose also will be determined by the existence, nature, and extent of any adverse side-effects that accompany the administration of a particular compound or vector in a particular patient

In determining the effective amount of the modulator to be administered in the treatment or prophylaxis of cancer, the physician evaluates circulating plasma levels of the modulator, modulator toxicities, progression of the disease, and the production of anti-modulator antibodies. In general, the dose equivalent of a modulator is from about 1 ng/kg to 10 mg/kg for a typical patient. Administration of compounds is well known to those of skill in the art (see, e.g., Bansinath et al., Neurochem Res. 18:1063-1066 (1993); Iwasaki et al., Jpn. J. Cancer Res. 88:861-866 (1997); Tabrizi-Rad et al., Br. J. Pharmacol. 111:394-396 (1994)).

For administration, modulators of the present invention can be administered at a rate determined by the LD-50 of the modulator, and the side-effects of the inhibitor at various concentrations, as applied to the mass and overall health of the patient. Administration can be accomplished via single or divided doses.

It must be noted that as used herein and in the appended claims, the singular forms “a”, “and”, and “the” include plural referents unless the context clearly dictates otherwise. Thus, for example, reference to “a nucleic acid” includes a plurality of such nucleic acids and reference to “the protein” includes reference to one or more proteins and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. Citations are incorporated herein by reference.

### EXAMPLES

### Example 1

### Generation of Prostate Specific Pten Null Mice

Experimental Procedures

Generation of Prostate Specific Pten Exon 5 Deletion Pten^{L/L}. C: Mice.

To generate Pten^{L/L};C⁺ mice, ARR2Probasin-Cre transgenic line, PB-Cre4 (Wu, X. et al., Mech. Dev. 101:61-69 (2001)) on C57BL/6×) BA2 background were crossed to Pten^{L/L} mice on a 129/Balb/c background. The males offspring with Pten^{L/+};C⁺ genotype were then crossed to Pten^{L/L} females. Only F2 generation of male offspring was used in this study.

Histology and Immunochemistry Analysis.

Tissues are fixed in 10% buffered Formalin for 6-10 hours, followed by gentle wash and transferred to 70% alcohol. These paraffin embedded tissues were sectioned (4 μm) and stained with Hematoxylin & Eosin. All IHC staining were performed on 4 μm sections that were prepared from paraffin-embedded blocks and placed on charged glass slides. Antigen retrieval was performed by incubating the slides in 0.01 M citric acid buffer (pH 6.0) at 95° C. for 30 minutes. Slides were then allowed to cool for 20 minutes in citric acid buffer. After washing in deionized water, the slides were then transferred to PBS (pH 7.4) (2×5 min each). The endogenous peroxidase activity was inactivated in a solution containing 3% hydrogen peroxide (H₂O₂) in methanol. The following detection and visualization procedures were performed according to manufacturer's protocol. Slides were counterstained in Mayer's hematoxylin, dehydrated, cleared and coverslipped. Negative control slides were run without primary antibody. Control slides known to be positive for each antibody were incorporated.

For AR (PG-21, Upstate Biotechnology), Nkx 3.1 (DE#2, a kind gift from Dr. Abate-Shen at the Center for Advanced Biotechnology and Medicine, Robert Wood Johnson Medical School) staining, pretreated sections were first blocked with 10% normal goat serum and then the primary antibody were diluted as suggested by the manufacture and incubated over night at 4° C. Following three washes with PBS, the antigens were visualized using the biotin-streptavidin based detection system from BioGenex. For clusterin-β (M−18, Santa Cruz Biotechnology) staining, the normal goat serum blocking was omitted.

For PTEN (26H9, Cell Signaling Technology) and P-AKT (#9277, Cell Signaling Technology) staining, pretreated sections were first blocked with mouse Ig blocking reagent in the VECTOR M.O.M. Immunodetection Kit (Vector Laboratories) and then incubated with primary antibody at room temperature for 30 min.

For fluorescence double staining, the section was treated as above and first stained with mouse antibody (PTEN) followed by signal amplification with TSA Plus Fluorescence Systems (PerkinElmer). After biotin blocking, the section was then stained with rabbit antibody (NKX 3.1, P-AKT) and signal was amplified with TSA system with different fluorescence.

Apoptosis and Proliferation Index

Cells undergoing apoptosis were determined by TUNEL assay using the In Situ Cell Death Detection Kit from Roche according to manufacture's instruction. Sections were de-waxed with xylene and rehydrated through graded alcohol. DNA fragmentation was labeled with fluorescein-conjugated dUTP and visualized with converter-POD and DAB. Apoptotic cell was identified by positive TUNEL staining and the appearance of apoptotic body. Five hundred cells were counted from 5 different view fields and the TUNEL positive cells were presented as numbers per 100 nucleated cell. Cell proliferation index was determined by Ki67 staining and calculated as above except 100 nucleated cells were counted per view field.

Western Blot Analysis

Extract was prepared by sonicating prostate tissues in buffer containing 50 mM Tris-HCl (pH 7.5), 150 mM NaCl, 0.5% NP-40, 1 mM EDTA, 1 mM PMSF and cocktail protease-inhibitors (Roche). 70 ug tissue lysate were subjected to SDS˜P AGE followed by Western blot analysis using anti-PTEN (9552, cell signaling), phospho-Akt (9271, cell signaling), NKX3.1 (a kind gift by Dr, Abate-Shen), PSCA (a kind gift by Dr. Owen Witte from UCLA), and actin (A 4700, sigma) antibodies, respectively.

Results

Cre-Mediated Pten Homozygous Deletion and Up-Regulated AKT Activity

To achieve Pten prostate-specific deletion, we crossed Pten^{loxp/loxp} mice (Lesche, R. et al., Genesis 32:148-149 (2002)) to the ARR2Probasin-Cre transgenic line, PB-Cre4, in which the Cre recombinase is under the control of a modified rat prostate-specific pro basin (PB) promoter (Wu, X. et al., Mech. Dev. 101:61-69 (2001)). By crossing PB-Cre4 to a conditional reporter mouse R26R, the original report indicated that Cre expression is specific for prostatic epithelial cells. However, its expression levels vary from lobe to lobe: highest in the lateral lobe (LP), followed by the ventral (VP), dorsal (DP), and least in anterior lobes (AP) (Wu, X. et al., Mech. Dev. 101:61-69 (2001)). Since Cre mediated recombination event is a unidirectional process, cells with Cre-mediated gene deletion are likely to increase and accumulate over time. Indeed, a recent follow up study indicated that Cre-mediated recombination events increased to near 100% in LP/VP/DP at the age of 8 months (Powell, W. C. et al., Curr. Drug Targets 4:263-279 (2003)).

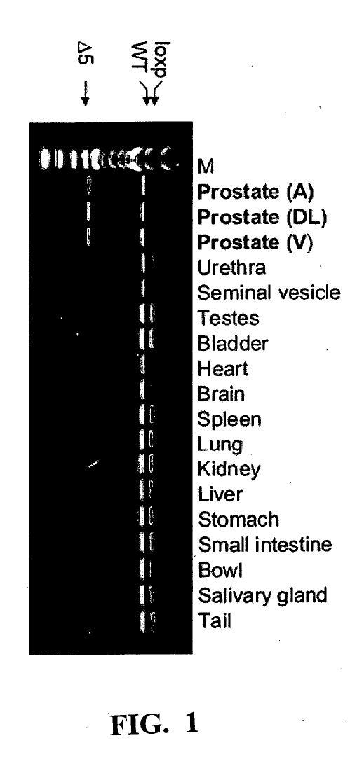

To confirm prostate-specific Pten deletion, the urogenital organs as well as other tissues from Pten^{loxp/+};Cre⁺ mice at the age of 9 and 25 weeks were carefully dissected and the status of Pten deletion were examined by sensitive PCR and immunohistochemistry (IHC) analyses. PCR analysis of 9 weeks old Pten^{loxp/+};PB-Cre⁴⁺ mice showed that Pten deletion, as indicated by excision of the exon 5 of Pten gene (Pten^{Δ5}), is specific to the prostate gland. Except trace amount of Pten ex on 5 deletion in the seminal vesicle, all the other tissues tested showed no detectable recombination activity (FIG. 1), consistent with the previous report (Wu, X. et al., Mech. Dev. 101:61-69 (2001)). Double immunofluorescent analysis demonstrated that PTEN is highly expressed in cytoplasm and in a less content in the nucleus of prostatic epithelial cells lining the prostatic acini as well as the stromal cells surrounding the acini (not shown). Deletion efficiencies of the Pten floxed alleles in different lobes were similar to the Cre expression pattern reported previously: PTEN immunostaining is significantly reduced in the lateral and ventral lobes but only lost or diminished in a subset of the cells in the dorsal and anterior lobes of 4-week-old Pten^{loxp/loxp};PB-Cre4⁺ mice (not shown). By 9 weeks, a majority of the cells in the epithelial compartment of DLP and VP show loss of PTEN immunostaining and approximately 40-60% cells in the AP are PTEN null (not shown). Furthermore, PTEN immunostaining in the stromal compartment remain positive, further confirming Pten epithelial-specific deletion.

As a result of PTEN loss, the AKT serine/threonine kinase, one of the primary targets of PTEN controlled signaling pathway, is activated. Thus, AKT phosphorylation and plasma membrane localization can serve as reliable indicators for PTEN loss. AKT phosphorylation is rarely detectable in the WT prostate but is highly expressed in Pten null cells, especially at the plasma membrane of the DLP and VP (not shown). Pten null, AKT-activated cells were also larger than their WT or heterozygous control cells, consistent with the role of PTEN in controlling cell size (Backman, S. et al., Curr Opin Neurobiol 12:516-522 (2002); Groszer, M. et al., Science 294:2186-2189 (2001)).

Pten Homozygous Deletion Shortens Latency for mPIN Formation

To determine whether deletion of both alleles at the Pten locus, or loss of heterozygosity (LOH), is required for prostate cancer initiation and progression, we examined cohorts of littermates, WT (Pten^{loxp/loxp};PB-Cre4⁻), heterozygous (Pten^{loxp/+};PB-Cre4⁺), and homozygous (Pten^{loxp/loxp};PB-Cre4⁺) for Pten prostate-specific deletion, from 4 to 29 weeks. To avoid potential variations contributed by genetic background, only mice from the F2 generation were used for studies described below.

Deletion of both alleles of Pten led to progressively enlarged prostate glands. Histological analysis indicated that from 4 weeks on the prostates of the mutant mice developed multi focal hyperplasia. These were initially observed in the dorsolateral and ventral lobes and subsequently involved the anterior lobes as well, consistent with the efficiency of Cre-mediated Pten deletion. Dorsolateral lobes from WT and their Pten null littermates from 4 to 12 weeks were compared. Epithelial hyperplasia, characterized by increased number of ce lls (without cellular atypia) is seen by 4 weeks of age (not shown). By 6 weeks of age, these mice develop murine PIN (mPIN). MPIN is the proliferation of atypical epithelial cells within pre-existing prostatic ductules and acini (not shown). This proliferation results in stratification of the epithelial layer, giving rise to distinctive architectural features, to include cribiform, tufting or micropapillary growth patterns. Cytological atypia is characterized by nuclear enlargement, nuclear contour irregularity, hyperchromatism, prominent nucleoli accompanied with the inversion of the nuclear to cytoplasmic ratio. 100% of the homozygous mice developed mPIN at 6 weeks (Table 1). Thus, homozygous Pten deletion significantly shortened the latency for MPIN formation from 8-10 months in heterozygous to 1.5 months in homozygous conditional knock-outs. Significantly, Pten nun mPIN lesions progress to invasive and metastatic cancers (see below). These results demonstrate that 1) homozygous PTEN loss alone is sufficient for prostate cancer initiation; and 2) Pten LOH is a rate-limiting step for prostate cancer initiation and progression.

TABLE 1
Phenotypes associate with Pten prostate-specific deletion

| Age (Weeks) | WT/Het | Homozygous |
|---|---|---|
| 4 | 2/2 normal | 4/4 hyperplasia |
| 6 | 15/15 normal | 9/9 PIN |
| 9-29 | 31/31 normal | 16/16 invasive carcinoma |
| 12-29 | | 5/11 with metastasis |
| | | 3/14 dead |

Homozygous Pten Deletion Leads to Invasive Adenocarcinoma

While mice with heterozygous Pten deletion develop mPIN late in their life (12-16 months), mice with homozygous Pten deletion develop invasive adenocarcinoma by 9 weeks of age. Adenocarcinoma was seen in all lobes. Dorsolateral lobes of WT and mutant prostate were compared at 9 and 12 weeks. From 9 weeks onwards, we have observed the extension of malignant cells, either as individual cells or as nests of acini, initially through the basement membrane, and subsequently the fibromuscular layer as shown by the loss of SMA immunostaining, invading into the stroma. This in turn induces both an inflammatory and a desmoplastic response. This desmoplastic response is characterized by focal stromal cellularity, found in association with the invasive cancer. These prostate cancer cells show increased proliferation compared to the WT controls, as indicated by their Ki67 positive staining (not chown).

Prostate epithelium includes basal cells, luminal cells and neuroendocrine cells. Transgenic mice overexpressing the oncogenic SV40 T antigen (Garabedian, E. M. et al., Proc Natl Acad Sci USA 95:15382-15387 (1998); Greenberg, N. M. et al., Proc Natl Acad Sci USA 92:3439-3443 (1995); Kasper, S. et al., Lab Invest 78:319-333 (1998); Masumori, N. et al., Cancer Res. 61:2239-2249 (2001)) develop both adenocarcinoma and neuroendocrine carcinoma. To define the origin of Pten null prostate cancers in the current model, we performed immunohistochemical analyses. Neuroendocrine cells in normal and neoplastic prostate are devoid of androgen receptor (AR) and are positive for chromogranin A and synaptophysin (SNP) (for review, see (Sciarra, A. et al., BJU Int. 91:438-445 (2003)). Pten null cancer cells were AR-positive, a hallmark of secretory epithelium, but were negative for the neuroendocrine cell marker synaptophysin. Thus, Pten deletion results in an adenocarcinoma, i.e., epithelial origin, differing from T antigen transgenic mice in which tumors are of neuroendocrine origin.

Homozygous Pten Deletion Leads to Metastatic Prostate Cancers

Similar to the progression of human prostate cancers, Pten null prostate cancers also progress from mPIN to invasive adenocarcinoma, then to metastatic carcinoma with precisely defined kinetics. We have observed lymphovascular invasion in the Pten conditional knock out mice from 12 weeks of age (present in 5 of 11 mice, Table 1) with subsequent seeding of the subcapsular sinuidal regions of draining lymph nodes (2 of 11 mice) and pulmonary alveolar spaces (3 of 11 mice). The metastatic tumor cells in the lung alveolar space remain AR positive and are negative for PTEN immunostaining. Thus, the conditional Pten null mouse represents the first animal model in which deletion of an endogenous gene leads to metastatic prostate cancer.

Pten Null Prostate Tumors do Respond to Castration

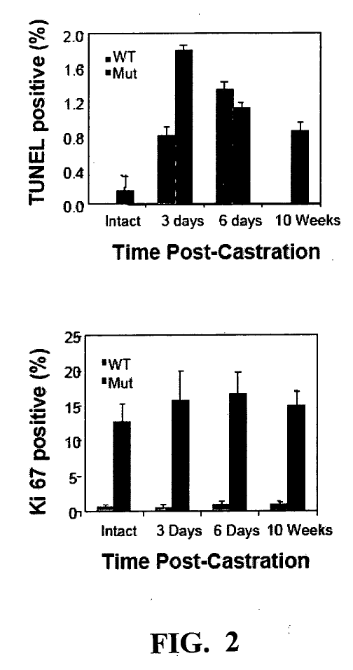

Androgens are critical both for development and function of the normal prostate gland and for the survival and proliferation of prostate cancer cells. To assess the response of Pten null prostate cancers to hormone ablation therapy, we castrated Pten conditional knock-out mice at 16 weeks, when invasive adenocarcinoma has already formed, and analyzed the immediate response of Pten null tumors at 3 and 6 days post castration. In response to androgen withdrawal, the AR positive prostatic epithelium undergoes increased apoptosis, as indicating by dramatically increased TUNEL positive cells, result in a reduction of prostate volume following castration (FIG. 2). In the WT control prostate, cell death can be easily detected 3 days after castration and peaks around 6 days (FIG. 2, top). Even though PTEN is known for its role in negatively regulating apoptosis (Di Cristofano, A. et al., Nat Genet 19:348-355 (1998)), quantitative analysis indicates that there is almost 10 times increase of apoptotic cells in the Pten null prostate 3 days post-castration compared with intact animal (FIG. 2, top; p<0.005), suggesting the survival of Pten null prostate cancer cells is androgen-dependent. The percentage of apoptotic cell drops when the measurement is taken at 6 days post castration (FIG. 2, top, p<0.005), indicating that Pten null prostate cancer cells may adapt to the new condition and exhibit enhanced survival, or the androgen sensitive population have been gradually depleted.

To test whether mice with Pten null prostate cancer would benefit from androgen ablation therapy, we castrated Pten conditional knock-outs at age of 2.5-4 months when invasive adenocarcinoma has already formed. For intact mice, 3/14 Pten prostate conditional knock-outs are died by the age of 12-29 weeks (Table 1). In contrast, no lethality is observed in 8/8 castrated Pten null mice aged from 7-10 months, indicating that Pten null prostate cancers do benefit from androgen ablation therapy. However, when these mice were sacrificed 2.5 months after castration, we found that a substantial number of Pten null prostate cancer cells remained. Histological analysis demonstrates that Pten null prostate glands remain 5-10-fold larger when compared to age-matched WT controls (not shown). Residual invasive adenocarcinoma is clearly evident (not shown). This enlargement, at least in part, is due to the higher proliferation index in the Pten null prostate. FIG. 2, bottom, shows Ki67 staining and quantification. Surprisingly, the proliferation indexes of Pten null prostate is 17-fold higher than age- and genetic background-matched WT controls at 3 day, as well as 6 days and 10 weeks after castration, and are comparable to the pre-castration stage, suggesting Pten deletion leads to androgen-independent or semi-independent cell proliferation. Interestingly, while most of the Pten null prostate cancer cells remain AR positive, it exhibits a more diffuse, heterogeneous immuno staining pattern (not shown). The Pten null prostate cancer cells are likely to be sensitive to androgen, as indicated by the higher percentage of TUNEL positive cells found in Pten null prostate 10 weeks after castration. Even though the remaining adenocarcinoma in Pten conditional knockouts did not lead to premature death during the short observation period, they may have the potential, as indicated by their ability to proliferate in the absence of androgen, to develop into HRPC, similar to that of humans, after prolonged castration.

### Example 2

### Identification of Prostate Cancer Biomarkers

Experimental Procedures

Microarray Preparation

Custom cDNA microarrays enhanced for genes expressed in the mouse prostate were prepared on poly-lysine-coated glass microscope slides using a robotic spotting tool as previously described (Aaltomaa, S. et al., Prostate 38:175-182 (1999).). Each array consisted of 10290 unique mouse cDNAs, 4511 of which were derived from cDNA libraries of developing and mature mouse prostate (www.mpedb.org) (Nelson, P. S. et al., Nucleic Acids Res. 30:218-220 (2002)). The remaining 5779 cDNAs were chosen from the Research Genetics sequence-verified set of IMAGE clones (www.resgen.com/products/SVMcDNA.php3) and from the National Institute of Aging 15K set. The clone inserts were amplified by PCR, purified, and analyzed by gel electrophoresis. All PCR products were sequence-verified prior to spotting. Additional control cDNAs were included and some clones were spotted twice for a total of 11552 features on the array.

Probe Construction, Microarray Hybridization, and Data Acquisition

The protocol used for indirect labeling of cDNAs was described previously (Pritchard, C. C. et al., Proc. Natl. Acad. USA 98:13266-13271 (2001)). Briefly, cDNA probes that incorporate aminoallyl dUTP (aa-dUTP; Sigma Aldrich) were made using 30 μg of total RNA. Purified cDNA from Pten null and age matched wild type prostates was labeled with either Cy3 or Cy5 mono-reactive fluors (Amersham Life Sciences), combined, and competitively hybridized to microarrays under a coverslip for 16 h at 63° C. Fluorescent array images were collected for Cy3 and Cy5 emissions using a GenePix 4000B fluorescent scanner (Axon Instruments, Foster City, Calif.). Image intensity data were extracted and analyzed using GenePix 4.0 microarray analysis software. RNA from 4 Pten null prostates and 4 age-matched wild type prostates was analyzed using the microarray. Each experiment was performed in duplicate with reversal of the fluorescent label to account for dye effects.

Data Normalization and Statistical Analysis

Log₂-ratios of PTEN/WT signal were normalized using a print-tip specific intensity based scatter plot smoother which uses robust locally linear fits to capture the dependence of the log-ratios on overall log-spot intensities (Dudoit, S. et al., Technical Report (Department of Biostatistics, University of California at Berkeley) (2000)). Statistical Analysis of Microarrays (SAM) software was used to determine genes that showed statistically significant differences in Pten null mice (Tusher, V. G. et al., Proc. Natl. Acad. Sci. USA 98:5116-5121 (2001)). At a delta of 2.36, 579 genes were significantly upregulated in Pten null prostates and 462 were downregulated. The median false discovery rate (FDR) was 0.085%, which predicts that ˜1 of the 1041 differentially expressed genes is falsely discovered. The differentially expressed genes other than ESTs were clustered and visualized with Cluster and TreeView program from Dr. Eisen's laboratory and the top 50 most significant were presented.

Results

Gene Expression Analysis Revealed Similarities between Molecular Mechanisms Underlying Pten Null Murine Cancers and Human Prostate Cancers

To provide insights into the molecular events associated with prostate tumorigenesis, we compared gene-expression profiles of Pten null prostates with age-matched WT controls using microarray analysis. Our initial studies were focused on animals 26-29 week-of age; since 100% of mutant animals at this stage have already developed invasive adenocarcinoma. Half of the prostate was fast frozen for RNA preparation and the other half was fixed for pathological evaluation. Histological analysis indicated that more than 80% of the Pten null prostate tissue at this stage was composed by microinvasive cancer cells and mPIN and less than 20 percent by stoma and inflammatory cells (data not shown). Statistical analysis of 10290 mouse genes/ESTs generated a list of 1041 significantly altered genes/ESTs. Among them, 579 are up-regulated in Pten null cancer and 462 are down-regulated, and the top 50 up- and down-regulated genes are shown in FIG. 3. The complete list of 1041 significantly altered genes/ESTs is shown in FIG. 4.

Gene expression changes in the Pten null prostate cancers included orthologues of genes whose expression also changes in human prostate cancers, such as up regulated cyclin A, clusterin, PSCA, S100P, ERG-1, and osteopontin, as well as down regulated Nk×3.1 and myosin heavy chain 11 (Aaltomaa, S. et al., Prostate 38:175-182 (1999); Bowen, C. et al., Cancer Res 60:6111-6115 (2000); Dhanasekaran, S. M. et al., Nature 412:822-826 (2001); Gu, Z. et al., Oncogene 19:1288-1296 (2000); He, W. et al., Genomics 43:69-77 (1997); Hotte, S. J. et al., Cancer 95:506-512 (2002); Mousses, S. et al., Cancer Res. 62:1256-1260 (2002); Ramaswamy, S. et al., Nat. Genet. 33:49-54 (2003); Reiter, R. E. et al., Proc. Natl. Acad. Sci. USA 95:1735-1740 (1998); Steinberg, J. et al., Clin. Cancer Res. Res. 3:1707-1711 (1997)). The corresponding protein expression levels of selected genes, Clu, PSCA, Nkx3, were further confimled by immunohistological staining or Western blot analysis. Some changes, such as Nkx3.1 and clusterin, were directly associated with homozygous Pten deletion and may be regulated by a PTEN controlled signaling pathway; other changes are observed during tumor progression, such as PSCA, or related to metastasis (osteopontin). These later groups may represent the additional genetic alterations associated with prostate cancer development. Clusterin (Steinberg, J. et al., Clin. Cancer Res. Res. 3:1707-1711 (1997)) and osteopontin (Hotte, S. J. et al., Cancer 95:506-512 (2002)) are secreted molecules and could be used as potential biomarkers for cancer staging and molecular diagnostics.

Recently, molecular signatures of metastatic potential have been found within the bulk cell mass of primary tumors, suggesting that metastasis may be an intrinsic property inherited in the primary cancers (Ramaswamy, S. et al., Nat. Genet. 33:49-54 (2003); van't Veer, L. J. et al., Nature 415:530-536 (2002)). Interestingly, among 17 such “signature genes” identified in various human cancers, 3 genes, namely Co11α1, Co11α2, and Myh11, are also up- or down-regulated in our Pten prostate models, consistent with the metastasis potential of Pten prostate cancer cells described in this study. Taken together, our initial characterization of changes in gene expression profiling suggests that the Pten prostate model will be useful to provide insights into the molecular events associated with prostate cancer progression and metastasis and to elucidate biomarkers and drug targets for clinical classification and therapeutic intervention.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A transgenic postnatal mouse that comprises a Pten-null prostate cell, wherein the Pten-null prostate cell comprises a genome comprising a homozygous disruption of the Pten gene, and wherein the Pten-null prostate cell has decreased levels of functional PTEN protein as compared to a prostate cell from a non-transgenic post-natal mouse.

2. A method of making a transgenic postnatal mouse of claim 1, the method comprising the steps of
a) crossing a first mouse comprising a Pten nucleic acid construct with a second mouse comprising a prostate-specific inducer of site-specific recombination, wherein the Pten nucleic acid construct comprises a Pten nucleic acid comprising specific recombination sites, and wherein, in the absence of recombination, the Pten nucleic acid expresses a functional PTEN protein; and
b) identifying progeny that have a prostate-specific homozygous disruption of the Pten gene and decreased expression of functional PTEN protein in prostate cells.

3. The method of claim 2, wherein the Pten nucleic acid construct comprises loxP sites that flank a region of the genomic Pten nucleic acid and the inducer of site-specific recombination comprises a Cre nucleic acid under the control of a prostate specific promoter.

4. The method of claim 3, wherein the loxP sites flank exon 5 of the genomic Pten nucleic acid.

5. The method of claim 3, wherein the prostate specific promoter is a probasin promoter.

6. A method of stimulating the deregulated growth of prostate cells in a mouse, the method comprising:
a. generating a transgenic postnatal mouse that comprises a Pten-null prostate cell, wherein the Pten-null prostate cell comprises a genome comprising a homozygous disruption of the Pten gene, and wherein the Pten-null prostate cell has decreased levels of functional PTEN protein as compared to a prostate cell from a non-transgenic post-natal mouse; and
b. allowing the transgenic mouse to grow for a time sufficient to permit detection of prostate cell hyperplasia.

7. The method of claim 6, further comprising the step of allowing the mouse to grow for a time sufficient to permit the detection of prostatic intraepithelial neoplasia (PIN).

8. The method of claim 6, further comprising the step of allowing the mouse to grow for a time sufficient to permit the detection of invasive adenocarcinoma of the prostate.

9. The method of claim 6, further comprising the step of allowing the mouse to grow for a time sufficient to permit the detection of metastatic prostate cancer.

10. The method of claim 6, further comprising the step of allowing the mouse to grow for a time sufficient to permit the detection of androgen independent cancer cells.

11. A method for assessing the effect of a composition or treatment on prostate cancer, the method comprising:
a. transgenic postnatal mouse that comprises a Pten-null prostate cell, wherein the Pten-null prostate cell comprises a genome comprising a homozygous disruption of the Pten gene, and wherein the Pten-null prostate cell has decreased levels of functional PTEN protein as compared to a prostate cell from a non-transgenic post-natal mouse;
b. allowing the mouse to grow for a time sufficient to permit the detection of prostate cancer;
c. applying the composition or treatment to the mouse; and
d. determining the effect of the composition or treatment on prostate cancer in the mouse.

12. The method of claim 11, wherein the mouse is allowed to grow for a time sufficient to permit the detection of prostatic intraepithelial neoplasia (PIN), and further comprising a step of determining the effect of the composition or treatment on PIN.

13. The method of claim 11, wherein the mouse is allowed to grow for a time sufficient to permit the detection of invasive adenocarcinoma, and further comprising a step of determining the effect of the composition or treatment on invasive adenocarcinoma in the mouse.

14. The method of claim 11, wherein the mouse is allowed to grow for a time sufficient to permit the detection of metastatic prostate cancer, and further comprising a step of determining the effect of the composition or treatment on metastatic prostate cancer in the mouse.

15. A method for assessing the effect of a composition or treatment on androgen independent prostate cancer, the method comprising:
a. generating a transgenic postnatal mouse that comprises a Pten-null prostate cell, wherein the Pten-null prostate cell comprises a genome comprising a homozygous disruption of the Pten gene, and wherein the Pten-null prostate cell has decreased levels of functional PTEN protein as compared to a prostate cell from a non-transgenic post-natal mouse;
b. allowing the mouse to grow for a time sufficient to permit the detection of an androgen independent prostate cancer cell;
c. applying the composition or treatment to the mouse; and
d. determining the effect of the composition or treatment on the androgen independent prostate cancer cells.

16. The method of claim 15, wherein the mouse is subjected to an androgen ablation therapy.

17. The method of claim 16, wherein the androgen ablation therapy is surgical.

18. The method of claim 16, wherein the androgen ablation therapy is chemical.

19. A method for identifying a prostate cancer biomarker, the method comprising:
a. transgenic postnatal mouse that comprises a Pten-null prostate cell, wherein the Pten-null prostate cell comprises a genome comprising a homozygous disruption of the Pten gene, and wherein the Pten-null prostate cell has decreased levels of functional PTEN protein as compared to a prostate cell from a non-transgenic post-natal mouse;
b. allowing the mouse to grow for a time sufficient to permit the detection of prostate cancer;
c. comparing an expression profile of a biological sample from the transgenic postnatal mouse to the expression profile of a biological sample from a control postnatal mouse; and
d. identifying differences in the expression profile that occur in the transgenic postnatal mouse relative to the control mouse, thereby identifying a prostate cancer biomarker.

20. The method of claim 19, wherein the mouse is allowed to grow for a time sufficient to permit the detection of prostatic intraepithelial neoplasia (PIN) in the transgenic postnatal mouse, and further comprising the steps of
c. comparing an expression profile of a biological sample comprising PIN from the transgenic postnatal mouse to the expression profile of a biological sample from a control postnatal mouse; and
d. identifying differences in the expression profile that occur in PIN in the transgenic postnatal mouse relative to the control mouse, thereby identifying a prostate cancer biomarker.

21. The method of claim 19, wherein the mouse is allowed to grow for a time sufficient to permit the detection of invasive adenocarcinoma, and further comprising the steps of:
c. comparing an expression profile of a biological sample comprising invasive adenocarcinoma from the transgenic postnatal mouse to the expression profile of a biological sample from a control postnatal mouse; and
d. identifying differences in the expression profile that occur in invasive adenocarcinoma in the transgenic postnatal mouse relative to the control mouse, thereby identifying a prostate cancer biomarker.

22. The method of claim 19, wherein the mouse is allowed to grow for a time sufficient to permit the detection of metastatic prostate cancer, and further comprising the steps of:
c. comparing an expression profile of a biological sample comprising metastatic prostate cancer from the transgenic postnatal mouse to the expression profile of a biological sample from a control postnatal mouse; and
d. identifying differences in the expression profile that occur in metastatic prostate cancer in the transgenic postnatal mouse relative to the control mouse, thereby identifying a prostate cancer biomarker.

23. A method for identifying an androgen independent prostate cancer-biomarker, the method comprising:
a. transgenic postnatal mouse that comprises a Pten-null prostate cell, wherein the Pten-null prostate cell comprises a genome comprising a homozygous disruption of the Pten gene, and wherein the Pten-null prostate cell has decreased levels of functional PTEN protein as compared to a prostate cell from a non-transgenic post-natal mouse;
b. allowing the mouse to grow for a time sufficient to permit the detection of an androgen independent prostate cancer cell; and
c. comparing an expression profile of a biological sample from the transgenic postnatal mouse to the expression profile of a biological sample from a control postnatal mouse; and
d. identifying differences in the expression profile that occur in the transgenic postnatal mouse relative to the control mouse, thereby identifying the androgen independent prostate cancer biomarker.

24. The method of claim 23, wherein the mouse is subjected to an androgen ablation therapy.

25. The method of claim 24, wherein the androgen ablation therapy is surgical.

26. The method of claim 24, wherein the androgen ablation therapy is chemical.

27. A Pten-null prostate cell, wherein a genome of the Pten-null prostate cell comprises a homozygous disruption of the Pten gene, and wherein the Pten-null prostate cell has decreased levels of functional PTEN protein as compared to a wild-type prostate cell.

28. The Pten-null prostate cell of claim 27, wherein the Pten-null prostate cell survives in the absence of androgens.
